SEQUENCE LISTING (2 sequences)

SEQ ID NO: 1 - DNA, 2850 bp, Homosapiens
tcgcacttccgtgtgccgcggcgccggagcccgaggcggctgtagcccacatctcccgagcgacccccggcgcccgcccgccgcgcggaggcccgggccacacctcactggccgcttggcccatcccagtcagcgccgcgccgaaccccgtccgcgcgcgccggggagcggcgcccccgccgctgccgccgcgacccttggcgcctgcccctgcaacgggaggtctgcagggaactggccaggcaagggggcaggcccgtttctcctggtggttggtgcgttgtagcagcagcgggagccaggactaaggacaagcaggagctgggagccccagccccactgcaggccccaccatggcgccgttcctgcgcatcgccttcaactcctatgagctgggctccctgcaggccgaggacgaggcgaaccagcccttctgtgccgtgaagatgaaggaggcgctcagcacagagcgtgggaaaacactggtgcagaagaagccgaccatgtatcctgagtggaagtcgacgttcgatgcccacatctatgaggggcgcgtcatccagattgtgctaatgcgggcagcagaggagccagtgtctgaggtgaccgtgggtgtgtcggtgctggccgagcgctgcaagaagaacaatggcaaggctgagttctggctggacctgcagcctcaggccaaggtgttgatgtctgttcagtatttcctggaggacgtggattgcaaacagtctatgcgcagtgaggacgaggccaagttcccaacgatgaaccgccgcggagccatcaaacaggccaaaatccactacatcaagaaccatgagtttatcgccaccttctttgggcaacccaccttctgttctgtgtgcaaagactttgtctggggcctcaacaagcaaggctacaaatgcaggcaatgtaacgctgccatccacaagaaatgcatcgacaagatcatcggcagatgcactggcaccgcggccaacagccgggacactatattccagaaagaacgcttcaacatcgacatgccgcaccgcttcaaggttcacaactacatgagccccaccttctgtgaccactgcggcagcctgctctggggactggtgaagcagggattaaagtgtgaagactgcggcatgaatgtgcaccataaatgccgggagaaggtggccaacctctgcggcatcaaccagaagcttttggctgaggccttgaaccaagtcacccagagagcctcccggagatcagactcagcctcctcagagcctgttgggatatatcagggtttcgagaagaagaccggagttgctggggaggacatgcaagacaacagtgggacctacggcaagatctgggagggcagcagcaagtgcaacatcaacaacttcatcttccacaaggtcctgggcaaaggcagcttcgggaaggtgctgcttggagagctgaagggcagaggagagtactttgccatcaaggccctcaagaaggatgtggtcctgatcgacgacgacgtggagtgcaccatggttgagaagcgggtgctgacacttgccgcagagaatccctttctcacccacctcatctgcaccttccagaccaaggaccacctgttctttgtgatggagttcctcaacgggggggacctgatgtaccacatccaggacaaaggccgctttgaactctaccgtgccacgttttatgccgctgagataatgtgtggactgcagtttctacacagcaagggcatcatttacagggacctcaaactggacaatgtgctgctggaccgggatggccacatcaagattgccgactttgggatgtgcaaagagaacatattcggggagagccgggccagcaccttctgcggcacccctgactatatcgcccctgagatcctacagggcctgaagtacacattctctgtggactggtggtctttcggggtccttctgtacgagatgctcattggccagtcccccttccatggtgatgatgaggatgaactcttcgagtccatccgtgtggacacgccacattatccccgctggatcaccaaggagtccaaggacatcctggagaagctctttgaaagggaaccaaccaagaggctgggagtgaccggaaacatcaaaatccaccccttcttcaagaccataaactggactctgctggaaaagcggaggttggagccacctttcaggcccaaagtgaagtcacccagagactacagtaactttgaccaggagttcctgaacgagaaggcgcgcctctcctacagcgacaagaacctcatcgactccatggaccagtctgcattcgctggcttctcctttgtgaaccccaaattcgagcacctcctggaagattgaggttcctggacagatcaggctagccctgccctccacccacacctgcccgctccccacgataagcaccagtgggactgtggtgacttctgctgctggccccgcccctgcccccagagcgtccttggctgccgtctggccgggctctcatggtacttcctctgtgaactgtgtgtgaatctgcttttcctctgccttcggagggaaattgtaaatcctgtgtttcattacttgaatgtagttatctattgaaaatatatattatatacatagacatatatatatatataataggctgtatatattgctcagtagagaaaaaccatgggggactggtgatatgttgatctttttcaaaaaaatatatatatgacaaaaaaaaaaaaaaaggagcacaagctgtttgaaccaccaggtttatttgtgtgtctaaataaacaccaaatagtaccaaaaaaaaaaaaaaaaa

SEQ ID NO: 2 - protein, 676 aa, Homosapiens
MAPFLRIAFNSYELGSLQAEDEANQPFCAVKMKEALSTERGKTLVQKKPTMYPEWKSTFDAHIYEGRVIQIVLMRAAEEPVSEVTVGVSVLAERCKKNNGKAEFWLDLQPQAKVLMSVQYFLEDVDCKQSMRSEDEAKFPTMNRRGAIKQAKIHYIKNHEFIATFFGQPTFCSVCKDFVWGLNKQGYKCRQCNAAIHKKCIDKIIGRCTGTAANSRDTIFQKERFNIDMPHRFKVHNYMSPTFCDHCGSLLWGLVKQGLKCEDCGMNVHHKCREKVANLCGINQKLLAEALNQVTQRASRRSDSASSEPVGIYQGFEKKTGVAGEDMQDNSGTYGKIWEGSSKCNINNFIFHKVLGKGSFGKVLLGELKGRGEYFAIKALKKDVVLIDDDVECTMVEKRVLTLAAENPFLTHLICTFQTKDHLFFVMEFLNGGDLMYHIQDKGRFELYRATFYAAEIMCGLQFLHSKGIIYRDLKLDNVLLDRDGHIKIADFGMCKENIFGESRASTFCGTPDYIAPEILQGLKYTFSVDWWSFGVLLYEMLIGQSPFHGDDEDELFESIRVDTPHYPRWITKESKDILEKLFEREPTKRLGVTGNIKIHPFFKTINWTLLEKRRLEPPFRPKVKSPRDYSNFDQEFLNEKARLSYSDKNLIDSMDQSAFAGFSFVNPKFEHLLED

(19) United States
(12) Patent Application Publication
(10) Pub. No.: US 2008/0069809 A1
(43) Pub. Date: Mar. 20, 2008

(54) Compositions and methods for the diagnosis and treatment of cancer

(76) Inventors: Steven Martin Ogbourne, Bunya (AU); Peter Gordon Parsons, St. Lucia (AU); Janet Lord, Birmingham (GB)

Correspondence Address: THE MCCALLUM LAW FIRM, P. C., 685 BRIGGS STREET, PO BOX 929, ERIE, CO, 80516, US

(21) Appl. No.: 11/786,957

(22) Filed: Apr. 13, 2007

Related U.S. Application Data
(60) Provisional application No. 60/794,043, filed on Apr. 20, 2006.

(51) Int. Cl. A61K 36/47; A61K 38/45; A61P 35/00; C12Q 1/48

(52) U.S. Cl. 424/94.5; 424/725; 435/15

(57) ABSTRACT

The present invention relates generally to the fields of diagnosis and treatment of cancer. More particularly, the present invention provides a method of determining the likely or relative sensitivity of cancer cells to a particular anti-cancer agent prior to or during anti-cancer treatment. Therapeutic protocols for the treatment of cancer based on a determination of the likely or relative sensitivity of the cancer cells to the anti-cancer agent also form part of the present invention. The present invention further contemplates rendering cancer cells sensitive or relatively more sensitive to an anti-cancer agent prior to or during anti-cancer treatment.

### CROSS REFERENCE TO RELATED APPLICATIONS

This application claims the benefit of U.S. Provisional Patent Application No. 60/794,043, filed Apr. 20, 2006, which is hereby incorporated by reference in its entirety.

### BACKGROUND OF THE INVENTION

1. Field of the Invention

The present invention relates generally to the field of oncology and treatment of cancer. More particularly, the present invention provides a method of determining the likely or relative sensitivity of cancer cells to a particular anti-cancer agent prior to or during anti-cancer treatment. Therapeutic protocols for the treatment of cancer based on a determination of the likely or relative sensitivity of the cancer cells to the anti-cancer agent also form part of the present invention. The present invention further contemplates rendering cancer cells sensitive or relatively more sensitive to an anti-cancer agent prior to or during anti-cancer treatment.

2. Description of the Prior Art

Natural product screening is a term applied to the screening of natural environments for bioactive molecules. Particularly sought after bioactive molecules are those having potential as useful therapeutic agents. Natural environments include plants, microorganisms, soil, coral and marine animals. The search for potential therapeutic agents for the treatment of cancer remains an important focus.

The Euphorbiaceae family of plants covers a wide variety of plants including weeds of Euphorbia species. One intensively studied species of this group is Euphorbia pilulifera L (synonyms E. hirta L., E. capitata Lam.), whose common names include pill-bearing spurge, snakeweed, cat's hair, Queensland asthma weed and flowery-headed spurge. The plant is widely distributed in tropical countries, including India, and in Northern Australia, including Queensland. Euphorbia peplus is another species from which ingenol angelates having anti-cancer properties has been isolated (See U.S. Pat. Nos. 6,432,452, 6,787,161 and 6,844,013).

The agent designated “PEP005” is an ingenol angelate (or angeloyl-substituted ingenane) extracted and purified from E. peplus, and is useful, inter alia in the treatment of actinic keratoses (AK) and non-melanoma skin cancer (NMSC) by short term topical administration. The cytotoxicity of PEP005 has been shown for many cell lines in vitro and its efficacy in vivo has been clinically established. The chemical name of PEP005 is ingenol-3-angelate (Ogbourne et al, Cancer Res 64:2833-2839, 2004).

Cancer is a cellular disease which occurs when a cell population hyper-proliferates. In the US alone, 2,604,650 people died from cancer between 1990-1994, with more men (53%) than women (47%) affected. The most numerous cancer deaths were the result of cancer of the lung (˜30%), colon and rectum (˜11%), breast (˜8%), and prostate (˜6.5%). Among women, the most commonly occurring cancers are breast (˜31%), lung (˜12%), colon and rectum (˜12%), uterus (˜6%) and ovary (˜4%). It is estimated that 570,280 people will die in the US from some form of cancer in 2005-2006. The occurrence of deaths for each type of cancer is expected to be in similar proportions as for those for 1990-1994.

Cancer treatment generally requires a therapeutic protocol comprising one or more of surgery, radiation and/or chemotherapy. Chemotherapy is a particularly common and well-established treatment for cancer. However, despite the fact that chemotherapeutic regimes provide significant benefit to patients, their use can be restricted because of problems with toxicity, adverse reactions and the emergence of chemoresistance. In fact, patients who are suffering from late-stage disease sometimes choose not to undergo active treatment because of the severe impact on the quality of life.

When treating malignant disease, low molecular weight (LMW) chemotherapeutic drugs demonstrate deficiencies in therapeutic efficacy, highlighting a growing need for a multi-disciplinary approach to cancer therapy (Jain, J Natl Cancer Inst 81:570, 1989; Jain, Science 271:1079, 1996). An understanding of aspects of drug selection including pharmacokinetics, pharmacodynamics, non-specific toxicity, chemoresistance, immunogenicity, biorecognition and efficacy is required in the development of more efficacious approaches to the treatment of cancer.

One particularly severe form of cancer is leukemia. Improvements in drug therapy and in patient care have progressively improved survival among younger patients with acute myeloid leukemia (AML). However, the risk of AML increases with age and success rates in older patients remain very poor (Schoch et al, Haematologica 89:1082-1090, 2004). As a result, the overall outcome remains very poor. In a study of 214 unselected AML patients in Sweden in whom the median age of patients was 69.5 years, median survival was 5.8 months and probable survival at five years was calculated as 9.3% (Astrom et al, Br J Cancer 82:1387-1392, 2000). The clinical problem of AML is exacerbated by the shifting population demographics in developed countries toward a more elderly population. Current estimates suggest that by the year 2050 approximately 40% of the population of Europe and North America will be older than 60 years (Lutz et al, Nature 387:803-805, 1997) and the impact of this statistic on AML and indeed other cancer incidence, and consequently on health care systems, is clear.

The factors underlying the poorer responses of elderly AML patients to chemotherapy are complex. However, they include the fact that AML occurring in older patients is less responsive to myelosuppressive chemotherapy and that older patients are intrinsically less able to tolerate this form of therapy. Circumvention of these obstacles will require the development of adjunctive therapies that improve tumor responses while not exacerbating the systemic toxicities of established chemotherapeutics.

A number of approaches can be used to find new drugs with possible application to AML and other cancers, including high throughput screening to small molecule libraries. An example of this is the identification of the Bcr-Abl selective protein kinase inhibitor imatinib (Glivec), which has been successfully exploited in the treatment of chronic myelogenous leukemia (CML) [Capdeville et al, Nature Rev Drug Discov. 1:493-502, 2002]. However, a potentially invaluable approach to identifying therapeutically useful small molecules is to employ naturally occurring molecules such as those from the Euphorbiaceae family.

PEP005 is a potent activator of protein kinase C (PKC) [Kedei et al, C. Cancer Res. 64:4243-3255, 2004], a family of signaling isoenzymes that regulate many cell processes including proliferation, differentiation and apoptosis (Deacon et al, J Clin Pathol Mol Pathol. 50:124-131, 1997; Mellor and Parker, Biochem J. 332:281-292, 1998). This signaling pathway has already been the target of several anti-cancer agents (Gescher, Crit Rev Oncol Hematol. 34:127-135, 2000; Clamp and Jayson, Anti-cancer Drugs 13:673-693, 2002; Han et al, Proc Natl Acad Sci USA 95:5357-5361, 1998). However, chemoresistance can develop to anti-cancer agents and some cancers are less sensitive to particular chemotherapeutic agents.

Hence, there is a need to determine what factors determine the sensitivity of cancer cells to particular anti-cancer agents.

### SUMMARY OF THE INVENTION

The present invention is predicated in part on a determination that the efficacy of particular anti-cancer agents is dependent on the level of a component of the PKC signaling pathway.

In particular, the anti-cancer agents are ingenol angelates, such as PEP005 and the component of the PKC signaling pathway is PKC-δ.

More particularly, the anti-leukemic activity of PEP005 was investigated against AML cells and primary AML blast cells. Data show that PEP005 was able to induce apoptosis in cell lines and primary AML blasts, whereas, in contrast, non-malignant myeloid blasts were resistant to PEP005-induced apoptosis but were induced to partially differentiate. Importantly, not all myeloid cell lines were equally sensitive to PEP005 and it was noted that the resistance displayed by KG1a cells was associated with the failure to express PKC-δ. However, transfection of KG1a cells with enhanced green fluorescent protein (EGFP)-PKC-δ restored not only PKC-δ expression but also an apoptotic response to PEP005. Analyses using selective PKC inhibitors and nuclear relocation further indicated that the anti-neoplastic action of PEP005 against AML is mediated via activation of PKC-δ. Hence, the efficacy of PEP005 in cancer cells is dependent on the presence or level of PKC-δ or other components in the PKC signaling pathway.

The present invention provides, therefore, a method for treating a subject with cancer, said cancer comprising cells which produce high levels of activities of a PKC or other component in the PKC signaling pathway said method comprising administering to said subject an anti-cancer effective amount of an ingenol angelate or pharmaceutically acceptable salt thereof.

The present invention further provides a method of treating a subject with cancer said method comprising determining the levels or activity of a PKC or other component in the PKC signaling pathway in one or more cancer cells and where the cancer cell or cells has or have low to zero levels of PKC or other component in the PKC signaling pathway contacting said cancer cells with the PKC or other component in the PKC signaling pathway or genetic means for generating same and an ingenol angelate or a pharmaceutically acceptable salt thereof.

Still another aspect of the present invention is directed to a method for treating a hematological-based cancer in a human subject, said method comprising contacting the leukemic cells with PKC or other component in the PKC signaling pathway and an ingenol angelate or a derivative, homolog, isomer or pharmaceutically acceptable salt thereof for a time and under conditions sufficient for the ingenol angelate to be toxic to said leukemic cells.

Even yet another aspect of the present invention provides for the use of an ingenol angelate and a PKC or genetic means for producing same in the manufacture of a medicament for the treatment of a subject having cancer.

The present invention also provides a diagnostic assay to determine the likely sensitivity of cancer cells to PEP005 or other ingenol angelates prior to or during anti-cancer treatment. Therapeutic protocols can be changed in response to suppression of PKC levels such as may occur in the development of chemoresistance. In addition, alternative therapies or addition of other agents may be indicated in subjects having low levels of PKC or other component in the PKC signaling pathway. Furthermore, where cancer tissue comprises cells with low levels of PKC, it is proposed, in one embodiment, to introduce PKC or the genetic apparatus to generate same in combination with the ingenol angelate such as PEP005.

Hence, the present invention contemplates a method for the presence, level or activity of PKC or other component in the PKC signaling pathway in cancer cells in a subject, said method comprising isolating the cancer cells and subjecting the cells to PKC-δ—or other component-detection means.

The present invention further provides a method for determining the sensitivity of a cancer cell or group of cancer cells to an ingenol angelate, said method comprising determining the level or activity of a PKC or other component in the PKC signaling pathway in said cancer cell or cells wherein the absence of low levels of PKC or other component compared to a control indicates that the cancer cell or cells is/are less sensitive to the ingenol angelate compared to cells with control levels of PKC.

The present invention provides, therefore, medical assessment protocols, methods of diagnosis, methods of treatment and therapeutic compositions useful in the treatment and monitoring of cancers in subjects.

The present invention still further provides a formulation comprising first and second parts wherein a first part comprises an ingenol angelate or a derivative, homolog, isomer or pharmaceutically acceptable salt thereof and a second part comprises a PKC or another component of the PKC signaling pathway or a genetic means of generating same.

A summary of the sequence identifiers used herein are shown in Table 1.

TABLE 1
Sequence Identifiers

| Sequence Identifier | Sequence |
|---|---|
| 1 | Human PKC-δ nucleotide sequence |
| 2 | Human PKC-δ amino acid sequence |

### BRIEF DESCRIPTION OF THE FIGURES

FIG. 1 is a graphical representation showing that PEP005 induces apoptosis and differentiation in leukemic cell lines according to an embodiment of the present invention. There were five leukemic cell lines treated with PEP005 for 5 days. (A-B) Differentiation was assessed by expression of CD11b and (C-D) apoptosis was assessed by entry of cells into a subdiploid phase. Differentiation and apoptosis were also measured in response to PEP005 and PMA after 3 days by assessing phagocytosis of yeast (E) and caspase-3 activity (F). Data in panels A and E are expression as mean±standard deviation.

FIG. 2 is a graphical representation showing that PEP005 synergizes with ATRA to induce CD11b expression according to an embodiment of the present invention. Of the lines, four were treated with 10 nM ATRA and 20 nM PEP005, alone or in combination and differentiation was determined after 5 days. Data are mean±SD of three experiments. *P<0.05, **P<0.01; and ***P<0.001 for data compared with ATRA alone.

FIGS. 3A through D are graphical and photographic representations showing PEP005 inducing apoptosis in primary AML marrow blasts but not in normal myeloblasts. (A) PEP005 induced apoptosis in a primary AML cell culture according to an embodiment of the present invention. Apoptosis was determined by appearance of a subdiploid peak (v) or active caspase-3 (λ). (B) Meaned data for apoptosis induction by PEP005 in seven primary AML samples. Data are mean±SD. *P<0.01 compared with non-parallel-treated controls. (C) Morphology on cytospins of example AML and non-malignant CD34⁺ myeloblasts from cord blood after treatment with 20 nM PEP005 and 200 nM PEP005, respectively. Differential staining showed that normal myeloblasts did not enter apoptosis, but when exposed to higher concentrations of PEP005 and a more differentiated phenotype. (D) An example of a FACS plot of non-malignant myeloblasts treated with PEP005, showing reduced CD34 staining indicative of differentiation.

FIGS. 4A through D are graphical and photographic representations showing that PEP005 actions are PKC-δ dependent according to an embodiment of the present invention. (A) Analysis by Western blotting of PKC-α, -β, -δ and -ξ expression in four leukemic cell lines. β-actin was assessed as a loading control. (B) Expression of PKC-δ correlated with PEP005 responsiveness. PKC-δ expression was assessed by Western blotting in four leukemic cell lines and measured by densitmetry. Data are expressed as PKC-δ expression relative to β-actin in the same sample. (C) Activation of PKC-δ by PEP005 (20 nM) and PMA (20 nM) was determined by measuring PKC-δ levels in the cytosolic (C), nuclear (N), and cell membrane (M) fractions of HL60 cells. (D) HL60 cells were incubated with 20 nM PEP005 for up to 9 hours and the presence of full-length (78 kDa) and the cleaved (40 kDa) fragment of PKC-δ was detected by Western blotting. (E) HL60 cells were incubated with 20 nM PEP005, 1 μM bisindolylmaleimide 1 (Bis-1), 20 nM Go6976 or PEP005 in combination with either Bis-1 or Go6976. Apoptosis was determined by caspase-3 activation. (F) HL60 cells were radiolabeled with ³²PO₄ prior to treatment with 20 nM PEP005 in the absence or presence of rottlerin and immunoprecipitation of caspase-3. An isotype-matched antibody (Irr) was used as a control. Blots in panels A, C and D are presentative of three separate experiments and data in panels B and E are mean±SD of three experiments.

FIGS. 5A through C are graphical and photographic representations showing transfection of cells with GFP-PKC-δ reinstated PEP005 responsiveness according to an embodiment of the present invention. (A) KG1a cells were transiently transfected with EGFP-tagged PKC-δ and expression of PKC-δ was confirmed by Western blotting after 24 hours. (B) PEP005 was added to cultures 24 hours after transfection and apoptosis was determined after 3 days in PEP005-treated EGFP-positive KG1a cells transfected with pEGFP empty vector (v) or pEGFP-PKC-δ (□) using analysis of forward and side scatter profiles by FACS. Data are mean±SD of 6 experiments. (C) Translocation of EGFP-tagged PKC-δ following treatment of KG1a cells with 200 nM PEP005 was monitored by confocal microscopy and shows translocation to the nuclear region of the cells within 5 minutes of treatment. Nuclei are counterstained with DAPI.

FIG. 6 is a diagram of components of PKC or PKC-like signaling pathways contemplated by the present invention.

### DETAILED DESCRIPTION OF THE INVENTION

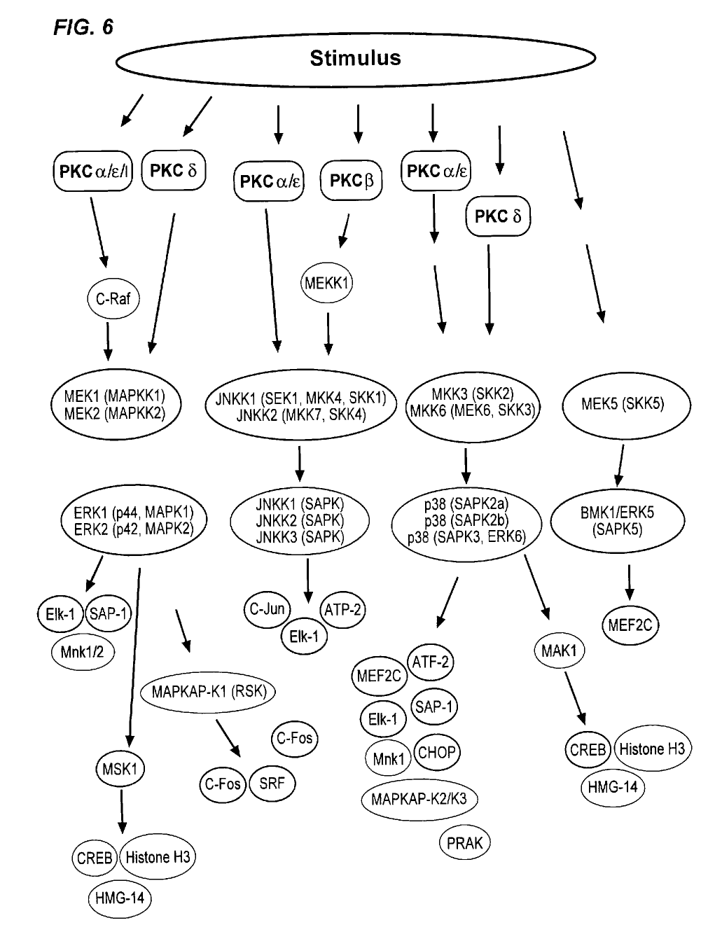

The present invention is predicated in part on the determination that ingenol angelates (angeloyl-substituted ingenanes) are more effective as cytotoxic agents in cells which produce PKC or isoform thereof or other component in the PKC or PKC-like signaling pathway. FIG. 6 summarizes the types of components contemplated by the present invention. Hence, the present invention enables the development of medical assessment protocols for the treatment of cancer by first determining whether the cancer cells produce PKC or a sufficient level or activity of PKC and then administering an angeloyl-substituted ingenane or a derivative, homolog or isomer thereof. Where cells do not produce PKC or the levels produced are such that the cell is rendered less sensitive to the ingenol angelate, the treatment involves the co-administration of the ingenol angelate and PKC or another component in the PKC signaling pathway or a genetic molecule encoding same. In a particular embodiment, the PKC isoform is PKC-δ.

Hence, one aspect of the present invention contemplates a method for treating a subject with cancer, said cancer comprising cells which produce high levels of activities of a PKC or other component in the PKC signaling pathway said method comprising administering to said subject an anti-cancer effective amount of an ingenol angelate or pharmaceutically acceptable salt thereof.

Another aspect of the present invention provides a method of treating a subject with cancer said method comprising determining the levels or activity of a PKC or isoform thereof or other component in the PKC signaling pathway in one or more cancer cells and where the cancer cell or cells has or have low to zero levels of PKC or isoform thereof or other component in the PKC signaling pathway contacting said cancer cells with the PKC, isoform or said other component in the PKC signaling pathway or genetic means for generating same and an ingenol angelate or a pharmaceutically acceptable salt thereof.

Some cancer types will characteristically have low levels of PKC-δ. In these instances, no determination need be made of PKC-δ levels.

Accordingly, another aspect of the present invention contemplates a method for treating a subject with cancer, said method comprising administering to said cancer an anti-cancer effective amount of an ingenol angelate and PKC or other component in the PKC signaling pathway or a genetic means for generating same for a time and under conditions sufficient to induce cytotoxicity to the cancer cells.

Preferably, the cancer is a hematological-based cancer. Hence, the present invention further contemplates a method for treating a hematological-based cancer in a human subject, said method comprising contacting the leukemic cells with PKC or other component in the PKC signaling pathway and an ingenol angelate or a derivative, homolog, isomer or pharmaceutically acceptable salt thereof for a time and under conditions sufficient for the ingenol angelate to be toxic to said leukemic cells.

The present invention still further contemplates, a method for determining the sensitivity of a cancer cell or group of cells such as cancer tissue to an ingenol angelate, said method comprising determining the level or activity of PKC-δ or other component in the PKC signaling pathway in said cancer cell or cells wherein the absence of or low levels of PKC-δ compared to a control indicates that the cancer cell or cells is/are less sensitive to the ingenol angelate compared to cells with control levels of PKC-δ.

Still another aspect of the present invention is directed to the use of an ingenol angelate and PKC-δ or other component in the PKC signaling pathway or a genetic means of generating same in the manufacture of a medicament for the treatment of a subject having cancer.

Hence, the present invention further provides for a pharmaceutical composition comprising at least two active components admixed together or maintained separately prior to delivery wherein a first component is an ingenol angelate or a derivative, homolog or isomer thereof and a second compound is PKC-δ or other component in the PKC signaling pathway or a genetic means for generating same.

The present invention provides, therefore, agents, reagents, compounds, pharmacologically active agents, medicaments, therapeutics, actives, and drugs in the form of an angeloyl-substituted ingenane or derivative, homolog or isomer thereof and PKC-δ or a genetic means for generating same.

As indicated above, in a particular embodiment the PKC or PKC isoform is PKC-δ.

In considering the present invention, the following terms or clarification need to be considered.

Throughout this specification, unless the context requires otherwise, the word “comprise”, or variations such as “comprises” or “comprising”, are understood to imply the inclusion of a stated element or integer or group of elements or integers but not the exclusion of any other element or integer or group of elements or integers.

All scientific citations, patents, patent applications and manufacturer's technical specifications referred to hereinafter are incorporated herein by reference in their entirety.

Reference to any prior art in this specification is not, and should not be taken as, an acknowledgment or any form of suggestion the prior art forms part of the common general knowledge in any country.

It is to be understood that unless otherwise indicated, the subject invention is not limited to specific formulation components, manufacturing methods, biological materials or reagents, dosage regimens and the like, as such may vary. It is also to be understood that the terminology used herein is for the purpose of describing particular embodiments only and is not intended to be limiting.

As used in the subject specification, the singular forms “a”, “an” and “the” include plural aspects unless the context clearly dictates otherwise. Thus, for example, reference to “a formulation” includes a single formulation, as well as two or more formulations; reference to “an agent” or “a reagent” includes a single agent or reagent, as well as two or more agents or reagents; reference to “the cancer cell” includes a single cancer cell or group or tissue of cancer cells; and so forth.

The terms “agent”, “reagent”, “compound”, “pharmacologically active agent”, “medicament”, “therapeutic”, “active” and “drug” are used interchangeably herein to refer to a chemical or genetic entity which induces or exhibits a desired effect such as generating PKC-δ or a component in the PKC signaling pathway and/or inducing toxicity to cells and in particular cancer cells. The terms also encompass pharmaceutically acceptable and pharmacologically active ingredients of those active agents specifically mentioned herein. When the terms “agent”, “reagent”, “compound”, “pharmacologically active agent”, “medicament”, “therapeutic”, active” and “drug” are used, then it is to be understood that this includes the active entity per se as well as pharmaceutically acceptable, pharmacologically active salts, esters, amides, prodrugs, metabolites, analogs, and the like. Hence, for example, an “angeloyl-substituted ingenane” or “ingenol angelate” includes its pharmaceutically acceptable salts and functional derivatives, homologs and isomers thereof.

An “isomer” of an ingenol angelate includes a tautomer, rotomer or other homolog form which retain activity. The isoform of PKC is PKC-δ. Reference herein to “PKC-δ” includes other isoforms of components (such as those in FIG. 6) whose levels or activities can influence the efficacy of anti-cancer activity of the ingenol angelate.

Reference to an “agent”, “chemical agent”, “compound”, “pharmacologically active agent”, “medicament”, “therapeutic”, “active” and “drug” includes combinations of two or more active agents. A “combination” also includes multi-part such as a two-part composition where the agents are provided separately and given or dispensed separately or admixed together prior to dispensation. For example, a multi-part pharmaceutical pack may have two or more agents separately maintained. Hence, this aspect of the present invention includes combination therapy. Combination therapy includes the co-administration of an an ingenol angelate and PKC-δ or other component in the PKC signaling pathway, an agent which elevates levels of PKC-δ or the other component or a genetic molecule or agent that encodes, upon expression, PKC-δ or other component.

The terms “effective amount” and “therapeutically effective amount” of an agent as used herein mean a sufficient amount of the agent to provide the desired therapeutic or physiological or effect or outcome. Such an effect or outcome includes chemoablation and/or apoptosis of cancer cells. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. Generally, the agent or agents is/are given in an amount and under conditions sufficient to induce chemoablation and/or apoptosis of the cancer cells.

The effective amount is deemed the amount required to inhibit the growth or viability of cancer cells or to otherwise induce their apoptosis or induce cell death. Effective amounts include for the ingenol angelate from above 0.1 nM to about 200 μM such as 0.1, 0.2, 0.3, 0.4, 0.5, 0.6, 0.7, 0.8,0.9, 1 nM or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nM or 100, 150, 200, 250, 300, 350, 400, 450, 500, 550, 600, 650, 700, 750, 800, 850, 900, 950, 100 nM (1 μM) or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 μM, or fractional amounts inbetween the integer amounts. Effective amounts of between 0.2 nM and 20 μM are particularly useful. Amounts of PKC-δ or other component in the PKC signaling pathway are proposed to be similar to those listed above for the ingenol angelate. In any event, the effective amount is the combination of ingenol angelate and PKC-δ or component in PKC signaling pathway effective to induce cell death.

As indicated above, the ingenol angelate and PKC-δ or genetic means for generating PKC-δ or a component in the PKC signaling pathway may involve the co-administration of another anti-cancer agent or immunological agent. Examples of suitable anti-cancer agents include anti-metabolites, anti-tumor antibiotics, mitotic inhibitors, steroids, sex hormones, alkylating agents, nitrogen mustards, nitrosources, hormone agonists, and microtubule inhibitors.

Anti-metabolites interfere with the body's chemical processes, such as protein or DNA synthesis required for cell growth and reproduction. Anti-metabolite drugs can prevent cell division which is a requirement in cancer treatment. Examples include Azaserine, D-Cycloserine, Mycophenolic acid, Trimethoprim, 5-fluorouracil, capecitabine, methotrexate, gemcitabine, cytarabine (ara-C) and fludarabine.

Anti-tumor antibiotics interfere with DNA by stopping enzymes and mitosis or altering the membranes that surround cells. These agents work in all phases of the cell cycle. Thus, they are widely used for a variety of cancers. Examples of anti-tumor antibiotics include dactinomycin, daunorubicin, doxorubicin (Adriamycin), idarubicin, and mitoxantrone.

Mitotic inhibitors are plant alkaloids and other compounds derived from natural products. They can inhibit, or stop, mitosis or inhibit enzymes for making proteins needed for reproduction of the cell. These work during the M phase of the cell cycle. Examples of mitotic inhibitors include paclitaxel, docetaxel, etoposide (VP-16), vinblastine, vincristine, and vinorelbine.

Steroids are natural hormones and hormone-like drugs that are useful in treating some types of cancer (such as but not limited to lymphoma, leukemias and multiple myeloma) as well as other illnesses. When these drugs are used to kill cancer cells or slow their growth, they are considered chemotherapeutic drugs. They are often combined with other types of chemotherapy drugs to increase their effectiveness. Examples include prednisone and dexamethasone.

Sex hormones, or hormone-like drugs, alter the action or production of female or male hormones. They are used to slow the growth of breast, prostate, and endometrial (lining of the uterus) cancers, which normally grow in response to hormone levels in the body. Examples include anti-estrogens (tamoxifen, fulvestrant), aromatase inhibitors (anastrozole, letrozole), progestins (megestrol acetate), anti-androgens (bicalutamide, flutamide), and LHRH agonists (leuprolide, goserelin).

Alkylating agents work directly on DNA to prevent the cancer cell from reproducing. As a class of drugs, these agents are not phase-specific (in other words, they work in all phases of the cell cycle). These drugs are active against chronic leukemias, non-Hodgkin's lymphoma, Hodgkin's disease, multiple myeloma, and certain cancers of the lung, breast, and ovary. Examples of alkylating agents include busulfan, cisplatin, carboplatin, chlorambucil, cyclophosphamide, ifosfamide, dacarbazine (DTIC), mechlorethamine (nitrogen mustard), and melphalan.

Nitrogen mustard in the form of its crystalline hydrochloride it is used as a drug in the treatment of Hodgkin's disease, non-Hodgkin's lymphomas and brain tumors. Nitrogen mustards cause mutations in the genetic material of cells, thereby disrupting mitosis, or cell division. Cells vary in their susceptibility to nitrogen mustards, with rapidly proliferating tumor and cancer cells most sensitive; bone marrow, which produces red blood cells, is also sensitive, and depression of red blood cell production is a frequent side effect of nitrogen mustard therapy. The nitrogen mustards also suppress the immune response. Other types include the aromatic mustards melphalan and chlorambucil.

Nitrosoureas act in a similar way to alkylating agents. They interfere with enzymes that help repair DNA. These agents are able to travel to the brain so they are used to treat brain tumors as well as non-Hodgkin's lymphomas, multiple myeloma, and malignant melanoma. Examples of nitrosoureas include carmustine (BCNU) and lomustine (CCNU).

Examples of hormone agonists include Leuprolide (Lupron, Viadur, Eligard) such as for prostate cancer, Goserelin (Zoladex) for breast and prostate cancers and Triptorelin (Trelstar) for ovarian and prostate cancers and nafarelin acetate (Synarel).

Microtubule inhibitors include “Vinca” alkaloids, taxoids and benzimidazoles. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES).

Particular chemotherapeutic agents contemplated for use with an ingenol angelate include those listed in Table 2.

In addition to these anti-cancer agents, antibodies or agents to stimulate the immune system or cytotoxic CD8⁺ cells may also be administered.

Combination therapy is particularly useful when cancer cells are assayed during anti-cancer treatment and levels of PKC-δ are determined to be reduced or are in the process of reducing.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, emide, prodrug, derivative, homolog or isomer of a compound as provided herein is a salt, ester, amide, prodrug or derivative that this not biologically or otherwise undesirable.

“Treating” a subject may involve chemoablation/apoptosis of cancer cells alone or as part of further chemotherapy and/or immunological therapy. The terms “treating” and “treatment” include ameliorating a clinically symptomatic individual by reducing the level of cancer cells.

The “subject” as used herein refers to an animal, preferably a mammal and more preferably a primate including a lower primate and even more preferably a human who can benefit from the formulations and methods of the present invention. A subject regardless of whether a human or non-human animal may be referred to as an individual, patient, animal, host or recipient. The compounds and methods of the present invention have applications in human medicine, veterinary medicine as well as in general, domestic or wild animal husbandry. For convenience, an “animal” includes an avian species such as a poultry bird (including ducks, chicken, turkeys and geese), an aviary bird or game bird. The condition in a non-human animal may not be a naturally occurring but induced such as in an animal model. In one example an animal model, cancer cells or cancer tissue is/are introduced to a mouse or rat or other non-human animal and then the animal is treated as herein disclosed.

As indicated above, the preferred animals are humans, non-human primates such as marmosets, baboons, orangutangs, lower primates such as tupia, livestock animals, laboratory test animals, companion animals or captive wild animals. A human is the most preferred target. However, non-human animal models may be used.

Examples of laboratory test animals include mice, rats, rabbits, guinea pigs and hamsters. Rabbits and rodent animals, such as rats and mice, provide a convenient test system or animal model as do primates and lower primates. Livestock animals include sheep, cows, pigs, goats, horses and donkeys. Non-mammalian animals such as avian species, zebrafish, amphibians (including cane toads) and Drosophila species such as Drosophila melanogaster are also contemplated. Instead of a live animal model, a test system may also comprise a tissue culture system.

TABLE 2
Examples of therapeutic agents for use in combination
therapy for the treatment of cancer

Alkylating Agents:
Alkeran - Melphalan
Alkeran Injection -Melphalan
BiCNU- Carmustine
CeeNU - Lomustine
Cycloblastin - Cyclophosphamide
Endoxan (containing sodium chloride) - Cyclophosphamide
Endoxan (new formulation without sodium chloride) - Cyclophosphamide
liadel Implant - Carmustine
Holoxan - Ifosfamide
Leukeran - Chlorambucil
Muphoran - Fotemustine
Myleran - Busulfan
Temodal - Temozolomide
Thiotepa - Thiotepa
Antibiotic cytotoxics:
Adriamycin Solution - Doxorubicin hydrochloride
Blenamax - Bleomycin sulfate
Blenoxane - Bleomycin sulfate
Bleomycin Sulfate for Injection (DBL) - Bleomycin sulfate
Caelyx - Doxorubicin hydrochloride
Cosmegen - Dactinomycin
Daunorubicin Injection - Daunorubicin hydrochloride
DaunoXome - Daunorubicin hydrochloride
Docetaxel
Doxorubicin Hydrochloride Injection (DBL) - Doxorubicin hydrochloride
Doxorubicin Hydrochloride Injection USP - Doxorubicin hydrochloride
Epirubicin Hydrochloride Injection (DBL)- Epirubicin hydrochloride
Fludara- Fludarabine phosphate
Mitomycin C Kyowa- Mitomycin
Mitozantrone Injection - Mitozantrone hydrochloride
Novantrone- Mitozantrone hydrochloride
Onkotrone- Mitozantrone hydrochloride
Pharmorubicin- Epirubicin hydrochloride
Zavedos- Idarubicin hydrochloride
Antimetabolites:
Cytarabine (DBL) - Cytarabine
Cytarabine Injection - Cytarabine
Efudix - Fluorouracil (5 Fluorouracil)
Fluorouracil Injection BP - Fluorouracil
Fluorouracil Injection BP - Fluorouracil
Gemzar - Gemcitabine hydrochloride
Hycamtin - Topotecan hydrochloride
Hydrea - Hydroxyurea
Lanvis - Thioguanine
Ledertrexate - Methotrexate
Leunase - Colaspase
Leustatin - Cladribine
Methoblastin - Methotrexat
Methotrexate Injection and Tablets (DBL) - Methotrexate
Methotrexate Injection BP - Methotrexate
Puri-Nethol - Mercaptopurine
Tomudex - Raltitrexed
Xeloda - Capecitabine
Hormonal anti-neoplastic agents:
Anandron - Nilutamide
Androcur-100 - Cyproterone acetate
Arimidex - Anastrozole
Aromasin - Exemestane
Chem mart Tamoxifen - Tamoxifen citrate
Cosudex - Bicalutamide
Cyprone - Cyproterone acetate
Cyprostat-100 - Cyproterone acetate
Cytadren - Aminoglutethimide
Depo-Provera - Medroxyprogesterone acetate
Depo-Ralovera - Medroxyprogesterone acetate
Eulexin - Flutamide
Fareston - Toremifene citrate
Femara - Letrozole
Flutamin - Flutamide
Fugerel - Flutamide
Genox - Tamoxifen citrate
GenRx Tamoxifen - Tamoxifen citrate
healthsense Tamoxifen - Tamoxifen citrate
Lucrin - Leuprorelin acetate
Lucrin Depot - Leuprorelin acetate
Medroxyhexal - Medroxyprogesterone acetate
Megace - Megestrol acetate
Nolvadex, Nolvadex-D - Tamoxifen citrate
Procur - Cyproterone acetate
Provera - Medroxyprogesterone acetate
Ralovera - Medroxyprogesterone acetate
Sandostatin - Octreotide
Tamosin - Tamoxifen citrate
Tamoxen - Tamoxifen citrate
Tamoxifen Hexal - Tamoxifen citrate
Tamoxifen-BC - Tamoxifen citrate
Terry White Chemists Tamoxifen - Tamoxifen citrate
Zoladex 10.8 mg Implant - Goserelin acetate
Zoladex 3.6 mg Implant - Goserelin acetate
Other anti-neoplastic agents:
Anzatax Injection - Paclitaxel
Agrylin - Anagrelide hydrochloride
Amsidyl - Amsacrine
Camptosar - Irinotecan hydrochloride
Carboplatin Injection - Carboplatin
Carboplatin Injection (DBL) - Carboplatin
Cisplatin Injection - Cisplatin
Cisplatin Injection (DBL) - Cisplatin
D.T.I.C. - Dacarbazine
Dacarbazine for Injection (DBL) - Dacarbazine
Eloxatin - Oxaliplatin
Etopophos - Etoposide phosphate
Etoposide Injection - Etoposide
Etoposide Injection (DBL) - Etoposide
Glivec - Imatinib mesylate
Herceptin - Trastuzumab
Hexalen - Altretamine
Mabthera- Rituximab
Natulan- Procarbazine hydrochloride
Proleukin- Aldesleukin (rbe)
Sodium Iodide (131I) Capsules (Therapy)- Sodium iodide (131I)
Taxol - Paclitaxel
Taxotere - Docetaxel
Vepesid- Etoposide
Vesanoid- Tretinoin
Vumon- Teniposide
Vinca alkaloids:
Eldisine - Vindesine sulfate
Navelbine - Vinorelbine tartrate
Oncovin - Vincristine sulfate
Velbe - 21
Vinblastine Sulfate Injection (DBL) - Vinblastine sulfate
Vincristine Sulfate Injection - Vincristine sulfate
Vincristine Sulfate Injection (DBL) - Vincristine sulfate
Vinorelbine

Reference herein to “cancer” includes solid and blood borne cancers, leukemias, sarcomas and carcinomas. The present invention is particularly directed to hematological-based cancers (which includes blood borne cancers) such as but not limited to myeloid leukemias. The terms “cancer” and “tumor” may be used interchangeably throughout the subject specification. Examples of cancers which may be treated using the protocol of the present invention include but are not limited to ABL1 protooncogene, AIDS related cancers, acoustic neuroma, acute lymphocytic leukemia, acute myeloid leukemia (AML), adenocystic carcinoma, adrenocortical cancer, agnogenic myeloid metaplasia, alopecia, alveolar soft-part sarcoma, anal cancer, angiosarcoma, aplastic anemia, astrocytoma, ataxia-telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia (CML), colorectal cancers, cutaneous t-cell lymphoma, dermatofibrosarcoma-protuberans, desmoplastic-small-round-cell-tumor, ductal carcinoma, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anemia, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynacological cancers, hematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's-cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid-tumor-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloid leukemia, myeloproliferative disorders, nasal cancer, nasopharyngeal cancer, nephroblastoma, neuroblastoma, neurofibromatosis, nijmegen breakage syndrome, non-melanoma skin cancer (NMSC), non-small-cell-lung-cancer-(NSCLC), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, rare-cancers-and-associated-disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (SCLC), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, Waldenstrom's-macroglobulinemia or Wilms' tumor.

The present invention is particularly useful in the treatment of leukemias and other hematological-based cancers such as a myeloid leukemias including AML and CML.

Reference to an “ingenol angelate” or “angeloyl-substituted ingenane” includes compounds isolated from plants such as from a species of the family Euphorbiaceae as well as derivatives, homologs, isomers or functional equivalents thereof as well as chemically synthesized forms thereof as well as pharmaceutically acceptable salts thereof or stereoisomers thereof. The terms “ingenol angelate” or “angeloyl-substituted ingenane” are used interchangeably in this specification.

An ingenol angelate has the general formula set forth below (Formula I):

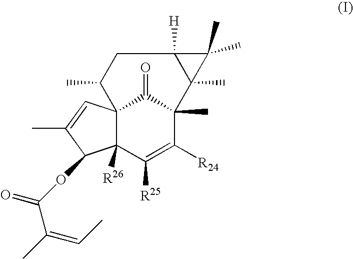

wherein:—
R₂₄, R₂₅ and R₂₆ are independently selected from hydrogen, hydroxy, R₂₇, R₂₈, F, Cl, Br, I, CN, OR₂₇, SR₂₇, NR₂₇R₂₈, N(═O)₂, NR₂₇OR₂₈, ONR₂₇R₂₈, SOR₂₇, SO₂R₂₇, SO₃R₂₇, SONR₂₇R₂₈, SO₂NR₂₇R₂₈, SO₃NR₂₇R₂₈, P(R₂₇)₃, P(═O)(R₂₇)₃, Si(R₂₇)₃, B(R₂₇)₂, (C═X)R₂₉ or X(C═X)R₂₉ where X is selected from sulfur, oxygen and nitrogen;
R₂₇ and R₂₈ are each independently selected from hydrogen, C₁-C₂₀ alkyl (branched and/or straight chained), C₁-C₂₀ arylalkyl, C₃-C₈ cycloalkyl, C₆-C₁₄ aryl, C₁-C₁₄ heteroaryl, C₁-C₁₄ heterocycle, C₂-C₁₀ alkenyl (branched and/or straight chained), C₂-C₁₀ alkynyl (branched and/or straight chained), C₁-C₁₀ heteroarylalkyl, C₁-C₁₀ alkoxyalkyl, C₁-C₁₀ haloalkyl, dihaloalkyl, trihaloalkyl, haloalkoxy, C₁-C₁₀ [CN, OR₂₇, SR₂₇, NR₂₇R₂₈, N(═O)₂, NR₂₇OR₂₈, ONR₂₇R₂₈, SOR₂₇, SO₂R₂₇, SO₃R₂₇, SONR₂₇R₂₈, SO₂NR₂₇R₂₈, SO₃NR₂₇R₂₈, P(R₂₇)₃, P(═O)(R₂₇)₃, Si(R₂₇)₃, B(R₂₇)₂]alkyl;
R₂₉ is selected from R₂₇, R₂₈, CN, COR₂₇, CO₂R₂₇, OR₂₇, SR₂₇, NR₂₇R₂₈, N(═O)₂, NR₂₇OR₂₈, ONR₂₇R₂₈, SOR₂₇, SO₂R₂₇, SO₃R₂₇, SONR₂₇R₂₈, SO₂NR₂₇R₂₈, SO₃NR₂₇R₂₈, P(R₂₇)₃, P(═O)(R₂₇)₃, Si(R₂₇)₃, B(R₂₇)₂.

Preferably, R₂₄ and R₂₅ are both hydroxy.

Reference herein to a member of the Euphorbiaceae family includes reference to species from the genera Acalypha, Acidoton, Actinostemon, Adelia, Adenocline, Adenocrepis, Adenophaedra, Adisca, Agrostistachys, Alchornea, Alchorneopsis, Alcinaeanthus, Alcoceria, Aleurites, Amanoa, Andrachne, Angostyles, Anisophyllum, Antidesma, Aphora, Aporosa, Aporosella, Argythamnia, Astrococcus, Astrogyne, Baccanrea, Baliospermum, Bernardia, Beyeriopsis, Bischofia, Blachia, Blumeodondron, Bonania, Bradleia, Breynia, Breyniopsis, Briedelia, Buraeavia, Caperonia, Caryodendron, Celianella, Cephalocroton, Chaenotheca, Chaetocarpus, Chamaesyce, Cheilosa, Chiropetalum, Choriophyllum, Cicca, Chaoxylon, Cleidon, Cleistanthus, Cluytia, Cnesmone, Cnidoscolus, Coccoceras, Codiaeum, Coelodiscus, Conami, Conceveiba, Conceveibastrum, Conceveibum, Corythea, Croizatia, Croton, Crotonopsis, Crozophora, Cubanthus, Cunuria, Dactylostemon, Dalechampia, Dendrocousinsia, Diaspersus, Didymocistus, Dimorphocalyx, Discocarpus, Ditaxis, Dodecastingma, Drypetes, Dysopsis, Elateriospermum, Endadenium, Endospermum, Erismanthus, Erythrocarpus, Erythrochilus, Eumecanthus, Euphorbia, Euphorbiodendron, Excoecaria, Flueggea, Calearia, Garcia, Gavarretia, Gelonium, Giara, Givotia, Glochidion, Clochidionopsis, Glycydendron, Gymnanthes, Gymnosparia, Haematospermum, Hendecandra, Hevea, Hieronima, Hieronyma, Hippocrepandra, Homalanthus, Hymenocardia, Janipha, Jatropha, Julocroton, Lasiocroton, Leiocarpus, Leonardia, Lepidanthus, Leucocroton, Mabea, Macaranga, Mallotus, Manihot, Mappa, Maprounea, Melanthesa, Mercurialis, Mettenia, Micrandra, Microdesmis, Microelus, Microstachy, Maocroton, Monadenium, Mozinna, Neoscortechinia, Omalanthus, Omphalea, Ophellantha, Orbicularia, Ostodes, Oxydectes, Palenga, Pantadenia, Paradrypeptes, Pausandra, Pedilanthus, Pera, Peridium, Petalostigma, Phyllanthus, Picrodendro, Pierardia, Pilinophytum, Pimeleodendron, Piranhea, Platygyna, Plukenetia, Podocalyx, Poinsettia, Poraresia, Prosartema, Pseudanthus, Pycnocoma, Quadrasia, Reverchonia, Richeria, Richeriella, Ricinella, Ricinocarpus, Rottlera, Sagotia, Sanwithia, Sapium, Savia, Sclerocroton, Sebastiana, Securinega, Senefeldera, Senefilderopsis, Serophyton, Siphonia, Spathiostemon, Spixia, Stillingia, Strophioblachia, Synadenium, Tetracoccus, Tetraplandra, Tetrorchidium, Thyrsanthera, Tithymalus, Trageia, Trewia, Trigonostemon, Tyria and Xylophylla.

In one embodiment, the genus and particularly suitable for the practice of the present invention is the genus Euphorbia. Particularly useful species of this genus include Euphorbia aaron-rossii, Euphorbia abbreviata, Euphorbia acuta, Euphorbia alatocaulis, Euphorbia albicaulis, Euphorbia algomarginata, Euphorbia aliceae, Euphorbia alta, Euphorbia anacampseros, Euphorbia andromedae, Euphorbia angusta, Euphorbia anthonyi, Euphorbia antiguensis, Euphorbia apocynifolia, Euphorbia arabica, Euphorbia ariensis, Euphorbia arizonica, Euphorbia arkansana, Euphorbia arteagae, Euphorbia arundelana, Euphorbia astroites, Euphorbia atrococca, Euphorbia baselicis, Euphorbia batabanensis, Euphorbia bergeri, Euphorbia bermudiana, Euphorbia bicolor, Euphorbia biformis, Euphorbia bifurcata, Euphorbia bilobata, Euphorbia biramensis, Euphorbia biuncialis, Euphorbia blepharostipula, Euphorbia blodgetti, Euphorbia boerhaavioides, Euphorbia boliviana, Euphorbia bracei, Euphorbia brachiata, Euphorbia brachycera, Euphorbia brandegee, Euphorbia brittonii, Euphorbia caesia, Euphorbia calcicola, Euphorbia campestris, Euphorbia candelabrum, Euphorbia capitellata, Euphorbia carmenensis, Euphorbia carunculata, Euphorbia cayensis, Euphorbia celastroides, Euphorbia chalicophila, Euphorbia chamaerrhodos, Euphorbia chamaesula, Euphorbia chiapensis, Euphorbia chiogenoides, Euphorbia cinerascens, Euphorbia clarionensis, Euphorbia colimae, Euphorbia colorata, Euphorbia commutata, Euphorbia consoquitlae, Euphorbia convolvuloides, Euphorbia corallifera, Euphorbia creberrima, Euphorbia crenulata, Euphorbia cubensis, Euphorbia cuspidata, Euphorbia cymbiformis, Euphorbia darlingtonii, Euphorbia defoliata, Euphorbia degeneri, Euphorbia deltoidea, Euphorbia dentata, Euphorbia depressa Euphorbia dictyosperma, Euphorbia dictyosperma, Euphorbia dioeca, Euphorbia discoidalis, Euphorbia dorsiventralis, Euphorbia drumondii, Euphorbia duclouxii, Euphorbia dussii, Euphorbia eanophylla, Euphorbia eggersii, Euphorbia eglandulosa, Euphorbia elata, Euphorbia enalla, Euphorbia eriogonoides, Euphorbia eriophylla, Euphorbia esculaeformis, Euphorbia espirituensis, Euphorbia esula, Euphorbia excisa, Euphorbia exclusa, Euphorbia exstipitata, Euphorbia exstipulata, Euphorbia fendleri, Euphorbia filicaulis, Euphorbia filiformis, Euphorbia florida, Euphorbia fruticulosa, Euphorbia garber, Euphorbia gaumerii, Euphorbia gerardiana, Euphorbia geyeri, Euphorbia glyptosperma, Euphorbia gorgonis, Euphorbia gracilior, Euphorbia gracillima, Euphorbia gradyi, Euphorbia graminea, Euphorbia graminiea Euphorbia grisea, Euphorbia guadalajarana, Euphorbia guanarensis, Euphorbia gymnadenia, Euphorbia haematantha, Euphorbia hedyotoides, Euphorbia heldrichii, Euphorbia helenae, Euphorbia helleri, Euphorbia helwigii, Euphorbia henricksonii, Euphorbia heterophylla, Euphorbia hexagona, Euphorbia hexagonoides, Euphorbia hinkleyorum, Euphorbia hintonii, Euphorbia hirtula, Euphorbia hirta, Euphorbia hooveri, Euphorbia humistrata, Euphorbia hypericifolia, Euphorbia inundata, Euphorbia involuta, Euphorbia jaliscensis, Euphorbia jejuna, Euphorbia johnston, Euphorbia juttae, Euphorbia knuthii, Euphorbia lasiocarpa, Euphorbia lata, Euphorbia latazi, Euphorbia latericolor, Euphorbia laxiflora Euphorbia lecheoides, Euphorbia ledienii, Euphorbia leucophylla, Euphorbia lineata, Euphorbia linguiformis, Euphorbia longecornuta, Euphorbia longepetiolata, Euphorbia longeramosa, Euphorbia longinsulicola, Euphorbia longipila, Euphorbia lupulina, Euphorbia lurida, Euphorbia lycioides, Euphorbia macropodoides, macvaughiana, Euphorbia manca, Euphorbia mandoniana, Euphorbia mangleti, Euphorbia mango, Euphorbia marylandica, Euphorbia mayana, Euphorbia melanadenia, Euphorbia melanocarpa, Euphorbia meridensis, Euphorbia mertonii, Euphorbia mexiae, Euphorbia microcephala, Euphorbia microclada, Euphorbia micromera, Euphorbia misella, Euphorbia missurica, Euphorbia montana, Euphorbia montereyana, Euphorbia multicaulis, Euphorbia multiformis, Euphorbia multinodis, Euphorbia multiseta, Euphorbia muscicola, Euphorbia neomexicana, Euphorbia nephradenia, Euphorbia niqueroana, Euphorbia oaxacana, Euphorbia occidentalis, Euphorbia odontodenia, Euphorbia olivacea, Euphorbia olowaluana, Euphorbia opthalmica, Euphorbia ovata, Euphorbia pachypoda, Euphorbia pachyrhiza, Euphorbia padifolia, Euphorbia palmeri, Euphorbia paludicola, Euphorbia parciflora, Euphorbia parishii, Euphorbia parryi, Euphorbia paxiana, Euphorbia pediculifera, Euphorbia peplidion, Euphorbia peploides, Euphorbia peplus, Euphorbia pergamena, Euphorbia perlignea, Euphorbia petaloidea, Euphorbia petaloidea, Euphorbia petrina, Euphorbia picachensis, Euphorbia pilosula, Euphorbia pilulifera, Euphorbia pinariona, Euphorbia pinetorum, Euphorbia pionosperma, Euphorbia platysperma, Euphorbia plicata, Euphorbia poeppigii, Euphorbia poliosperma, Euphorbia polycarpa, Euphorbia polycnemoides, Euphorbia polyphylla, Euphorbia portoricensis, Euphorbia portulacoides Euphorbia portulana, Euphorbia preslii, Euphorbia prostrata, Euphorbia pteroneura, Euphorbia pycnanthema, Euphorbia ramosa, Euphorbia rapulum, Euphorbia remyi, Euphorbia retroscabra, Euphorbia revoluta, Euphorbia rivularis, Euphorbia robusta, Euphorbia romosa, Euphorbia rubida, Euphorbia rubrosperma, Euphorbia rupicola, Euphorbia sanmartensis, Euphorbia saxatilis M. Bieb, Euphorbia schizoloba, Euphorbia sclerocyathium, Euphorbia scopulorum, Euphorbia senilis, Euphorbia serpyllifolia, Euphorbia serrula, Euphorbia setiloba Engelm, Euphorbia sonorae, Euphorbia soobyi, Euphorbia sparsiflora, Euphorbia sphaerosperma, Euphorbia syphilitica, Euphorbia spruceana, Euphorbia subcoerulea, Euphorbia stellata, Euphorbia submammilaris, Euphorbia subpeltata, Euphorbia subpubens, Euphorbia subreniforme, Euphorbia subtrifoliata, Euphorbia succedanea, Euphorbia tamaulipasana, Euphorbia telephioides, Euphorbia tenuissima, Euphorbia tetrapora, Euphorbia tirucalli, Euphorbia tomentella, Euphorbia tomentosa, Euphorbia torralbasii, Euphorbia tovariensis, Euphorbia trachysperma, Euphorbia tricolor, Euphorbia troyana, Euphorbia tuerckheimii, Euphorbia turczaninowii, Euphorbia umbellulata, Euphorbia undulata, Euphorbia vermiformis, Euphorbia versicolor, Euphorbia villifera, Euphorbia violacea, Euphorbia whitei, Euphorbia xanti Engelm, Euphorbia xylopoda Greenm., Euphorbia yayalesia Urb., Euphorbia yungasensis, Euphorbia zeravschanica and Euphorbia zinniiflora.

In a particular embodiment, the species of the genus Synadenium include Synadenium grantii and Synadenium compactum.

In a particular embodiment, the species of the genus Monadenium include Monadenium lugardae and Monadenium guentheri.

In a particular embodiment, the genus Endadenium is Endadenium gossweileni.

Euphorbia peplus is particularly useful in the practice of the present invention in terms of providing a source of ingenol angelates. Reference herein to “Euphorbia peplus” or its abbreviation “E. peplus” includes various varieties, strains, lines, hybrids or derivatives of this plant as well as its botanical or horticultural relatives. Furthermore, the present invention may be practiced using a whole Euphorbiaceae plant or parts thereof including sap or seeds or other reproductive material may be used. Generally, for seeds or reproductive material to be used, a plant or plantlet is first required to be propagated.

Reference herein to a Euphorbiaceae plant, a Euphorbia species or E. peplus further encompasses genetically modified plants. Genetically modified plants include trangenic plants or plants in which a trait has been removed or where an endogenous gene sequence has been down-regulated, mutated or otherwise altered including the alteration or introduction of genetic material which exhibits a regulatory effect on a particular gene. Consequently, a plant which exhibits a character not naturally present in a Euphorbiaceae plant or a species of Euphorbia or in E. peplus is nevertheless encompassed by the present invention and is included within the scope of the above-mentioned terms.

The angeloyl-substituted ingenanes are generally found in extracts of the Euphorbiaceae plants. An extract may comprise, therefore, sap or liquid or semi-liquid material exuded from, or present in, leaves, stem, flowers, seeds, bark or between the bark and the stem. Most preferably, the extract is from sap. Furthermore, the extract may comprise liquid or semi-liquid material located in fractions extracted from sap, leaves, stems, flowers, bark or other plant material of the Euphoriaceae plant. For example, plant material may be subject to physical manipulation to disrupt plant fibres and extracellular matrix material and inter- and intra-tissue extracted into a solvent including an aqueous environment. All such sources of ingenol angelates are encompassed by the present invention including compounds obtained by chemically synthetic routes.

In a particular embodiment, the compound of the present invention is referred to chemically as ingenol-3-angelate and is also referred to herein as “PEP005”. Reference herein to “ingenol-3-angelate” or “PEP005” includes naturally occurring as well as chemically synthetic forms and pharmaceutically acceptable salts, derivatives, homologs and isomers thereof.

Reference to “cytotoxic to cancer cells” or the induction of “apoptosis” of cancer cells or “chemoablation” is not to be construed that the therapy is selectively cytotoxic to only cancer cells although such selectivity is within the scope of the present invention.

As indicated above, the phrase “pharmaceutically acceptable salt, derivative, homologs or analogs” is intended to convey any pharmaceutically acceptable tautomer, salt, pro-drug, hydrate, solvate, metabolite or other compound which, upon administration to the subject, is capable of providing (directly or indirectly) the compound concerned or a physiologically (e.g. analgesically) active compound, metabolite or residue thereof. An example of a suitable derivative is an ester formed from reaction of an OH or SH group with a suitable carboxylic acid, for example C₁₋₃alkyl-CO₂H, and HO₂C—(CH₂)ₙ—CO₂H (where n is 1-10 such as 1, 2, 3, 4, 5, 6, 7, 8, 9, 10, but preferably 1-4), and CO₂H—CH₂phenyl.

Thus, the active compounds may be in crystalline form, either as free compounds or as solvates (e.g. hydrates). Methods of solvation are generally known within the art.

The salts of the active compounds of the subject invention may be pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulfuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulfphonic, toluenesulphonic, benzenesulphonic, salicyclic, sulphanilic, aspartic, glutamic, edetic, stearic, palmitic, oleic, lauric, pantothenic, tannic, ascorbic and valeric acids.

The term “pro-drug” is used herein in its broadest sense to include those compounds which can be converted in vivo to the compound of interest (e.g. by enzymatic or hydrolytic cleavage). Examples thereof include esters, such as acetates of hydroxy or thio groups, as well as phosphates and sulphonates. Processes for acylating hydroxy or thio groups are known in the art, e.g. by reacting an alcohol (hydroxy group), or thio group, with a carboxylic acid. Other examples of suitable pro-drugs are described in Bundgaard, Elsevier, Design of Prodrugs, 1985, the disclosure of which is included herein in its entirety by way of reference.

The term “metabolite” includes any compound into which the active agents can be converted in vivo once administered to the subject. Examples of such metabolites are glucuronides, sulphates and hydroxylates.

It will be understood that active agents as described herein may exist in tautomeric forms. The term “tautomer” is used herein in its broadest sense to include compounds capable of existing in a state of equilibrium between two isomeric forms. Such compounds may differ in the bond connecting two atoms or groups and the position of these atoms or groups in the compound. A specific example is keto-enol tautomerism.

The compounds of the present invention may be electrically neutral or may take the form of polycations, having associated anions for electrical neutrality. Suitable associated anions include sulfate, tartrate, citrate, chloride, nitrate, nitrite, phosphate, perchlorate, halosulfonate or trihalomethylsulfonate.

The active agents may be administered for therapy by any suitable route. Suitable routes of administration may include oral, rectal, nasal, inhalation of aerosols or particulates, topical (including buccal and sublingual), transdermal, vaginal, intravesical, intralesional and parenteral (including subcutaneous, intramuscular, intravenous, intrasternal, intrathecal, epidural and intradermal). Particularly useful forms of administration include a bolus administration to the tumor such as an intra-tumoral or intra-lesional application.

In one embodiment of the present invention, a PKC-δ or a component in the PKC signaling pathway to be present in order for the ingenol angelate to be most effective. The PKC-δ or other component may be administered directly to the cancer tissue or an agent may be employed which up-regulates expression of genetic material encoding the PKC-δ or other component or which down-regulates an inhibitor or PKC-δ or other signaling component.

In a particular embodiment, a genetic construct is employed which, when introduced to a cell, is expressed generating PKC-δ or the other component.

A genetic molecule may be introduced into a cell via a vector such that the nucleic acid sequence remains extrachromosomal. In such a situation, the nucleic acid sequence will be expressed by the cell from the extrachromosomal location. Vectors for introduction of nucleic acid sequence both for insertion into the chromosome or genome or for extrachromosomal maintenance are known in the art and any suitable vector may be used. Methods for introducing nucleic acids into cells such as electroporation, calcium phosphate co-precipitation and viral transduction are known in the art.

In particular, a number of viruses have been used as nucleic acid transfer vectors or as the basis for preparing nucleic acid transfer vectors, including papovaviruses (e.g. SV40, Madzak et al, J Gen Virol 73:1533-1536, 1992), adenovirus (Berkner, Curr Top Microbiol Immunol 158:39-66, 1992; Berkner et al, BioTechniques 6:616-629, 1988; Gorziglia and Kapikian, J Virol 66:4407-4412, 1992; Quantin et al, Proc Natl Acad Sci USA 89:2581-2584, 1992; Rosenfeld et al, Cell 68:143-155, 1992; Wilkinson et al, Nucleic Acids Res 20:233-2239, 1992; Stratford-Perricaudet et al, Hum Gene Ther 1:241-256, 1990; Schneider et al, Nat Genetics 18:180-183, 1998), vaccinia virus (Moss, Curr Top Microbiol Immunol 158:5-38, 1992; Moss, Proc Natl Acad Sci USA 93:11341-11348, 1996), adeno-associated virus (Muzyczka, Curr Top Microbiol Immunol 158:97-129, 1992; Ohi et al, Gene 89:279-282, 1990; Russell and Hirata, Nat Genetics 18:323-328, 1998), herpesviruses including HSV and EBV (Margolskee, Curr Top Microbiol Immunol 158:67-95, 1992; Johnson et al, J Virol 66:2952-2965, 1992; Fink et al, Hum Gene Ther 3:1-19, 1992; Breakefield and Geller, Mol Neurobiol 1:339-371, 1987; Freese et al, Biochem Pharmaco. 40:2189-2199, 1990; Fink et al, Ann Rev Neurosci 19:265-287, 1996), lentiviruses (Naldini et al, Science 272:263-267, 1996), Sindbis and Semliki Forest virus (Berglund et al, Biotechnology 11:916-920, 1993) and retroviruses of avian (Bandyopadhyay and Temin, Mol Cell Biol 4:749-754, 1984; Petropoulos et al, J Virol 66:3391-3397, 1992), murine (Miller, Curr Top Microbiol Immunol 158:1-24, 1992; Miller et al, Mol Cell Biol 5:431-437, 1985; Sorge et al, Mol Cell Biol 4:1730-1737, 1984; Mann and Baltimore, J Virol 54:401-407, 1985; Miller et al, J Virol 62:4337-4345, 1988) and human (Shimada et al, J Clin Invest 88:1043-1047, 1991; Helseth et al, J Virol 64:2416-2420, 1990; Page et al, J Virol 64:5270-5276, 1990; Buchschacher and Panganiban, J Virol 66:2731-2739, 1982) origin.

Non-viral nucleic acid transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated nucleic acid transfer can be combined with direct in vivo nucleic acid transfer using liposome delivery, allowing one to direct the viral vectors to particular cells. Alternatively, the retroviral vector producer cell line can be injected into particular tissue. Injection of producer cells would then provide a continuous source of vector particles.

The constructs of the present invention generally comprise a nucleic acid molecule encoding PKC-δ or other component of the PKC signaling pathway. Alternatively, the construct comprises an expression-inhibiting nucleic acid molecule which down-regulates an inhibitor of PKC-δ or other compound.

Reference to “PKC-δ” generally includes its homologs, polymorphic variants and mutated forms. The human form of PKC-δ is encoded by the nucleotide sequence comprising the sequence set forth in SEQ ID NO:1 or a variant or homolog thereof having at least about 60% identity thereto or which is capable of hybridizing to SEQ ID NO:1 or complementary form thereof under low stringency conditions. The human PKC-δ form comprises the amino acid sequence as set forth in SEQ ID NO:2 or a sequence having at least 60% similarity thereto after optimal alignment.

Having at least about “60% identity” or “60% similarity” means, having after optimal alignment, a nucleic acid molecule or amino acid sequence which comprises at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% identity or similarity with SEQ ID NO:1 or SEQ ID NO:2.

The terms “similarity” or “identity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and amino acid sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al (Nucl Acids Res 25:3389-3402, 1997). A detailed discussion of sequence analysis can be found in Unit 19.3 of Ausubel et al (Current Protocols in Molecular Biology John Wiley & Sons Inc, Chapter 15, 1994-1998).

The terms “sequence similarity” and “sequence identity” as used herein refer to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e. the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

Variants and homologs of the nucleic acid molecules of the present invention are also capable of hybridizing to other genetic molecules. Reference herein to “hybridizes” refers to the process by which a nucleic acid strand joins with a complementary strand through base pairing. Hybridization reactions can be sensitive and selective so that a particular sequence of interest can be identified even in samples in which it is present at low concentrations. Stringent conditions can be defined by, for example, the concentrations of salt or formamide in the prehybridization and hybridization solutions, or by the hybridization temperature, and are well known in the art. For example, stringency can be increased by reducing the concentration of salt, increasing the concentration of formamide, or raising the hybridization temperature, altering the time of hybridization. In alternative aspects, nucleic acids of the invention are defined by their ability to hybridize under various stringency conditions (e.g., high, medium, and low).

Reference herein to a “low stringency” includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as “medium stringency”, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or “high stringency”, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C)% (Marmur and Doty, J Mol Biol 5:109-118, 1962). However, the Tₘ of a duplex nucleic acid molecule decreases by 1° C. with every increase of 1% in the number of mismatch base pairs (Bonner and Laskey, Eur J Biochem 46:83-88, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

Reference to a “compound” of the PKC signaling pathway includes an up-stream or down-stream component relative to PKC-δ. Examples include MEKI(MAPKK1), MEK2(MAPKK2), ERK1(p44), ERK2(p42), Elk1, SAP-1, MnK1/2, MSK-1, CREB, Histone H3 and/or HMG14. p42 and p44 are particularly preferred components.

The present invention extends to assays to determine the level or activity of PKC-δ or other compound in the PKC signaling pathway. Assays contemplated herein include measuring amounts of PKC-δ protein, PKC-δ mRNA and enzymatic products of PKC-δ activity. In addition or alternatively, the amount of PKC-δ or its activity can be inferred from levels of proliferation, differentiation and/or extent of apoptosis.

Hence, another aspect of the present invention contemplates a method for screening for the presence, level or activity of PKC or other component in the PKC signaling pathway in cancer cells in a subject, said method comprising isolating the cancer cells and subjecting the cells to PKC-δ—or other component-detection means.

In a particular embodiment, the PKC is PKC-δ or a component as outlined in FIG. 6.

Generally, the cells are disrupted and the PKC-δ or other component measured by nucleic acid detection or protein detection assays. Examples of the former include PCR or primer/probe hybridization assays such as to screen for mRNA and the latter includes antibody assays, cellular assays or HPLC or other chromatography assays. Western blot assays for PKC-δ are particularly useful.

The assay for PKC-δ or other component may be conducted prior to chemotherapy with an ingenol angelate or during therapy to monitor for reduced responsiveness (i.e. chemoresistance) to the ingenol angelate.

Hence, this aspect of the present invention includes a medical assessment protocol to assist a clinician in determining whether or not to use an ingenol angelate, to use the ingenol angelate in combination with PKC-δ or other component in the PKC signaling pathway or to use additional chemotherapeutic agents in combination with the ingenol angelate.

The present invention also relates to compositions comprising an ingenol angelate or a pharmaceutically acceptable salt, derivative, homolog or isomer thereof, and PKC-δ or other component in the PKC signaling pathway or a genetic molecule encoding same together with one or more pharmaceutically acceptable additives and optionally other medicaments. The pharmaceutically acceptable additives may be in the form of carriers, diluents, adjuvants and/or excipients and may include all conventional solvents, dispersion agents, fillers, solid carriers, coating agents, antifungal or antibacterial agents, dermal penetration agents, surfactants, isotonic and absorption agents and slow or controlled release matrices. The active agents may be presented in the form of a kit of components adapted for allowing concurrent, separate or sequential administration of the active agents. Each carrier, diluent, adjuvant and/or excipient must be “pharmaceutically acceptable” in the sense of being compatible with the other ingredients of the composition and physiologically tolerated by the subject. The compositions may conveniently be presented in unit dosage form and may be prepared by methods well known in the art of pharmacy. Such methods include the step of bringing into association the active ingredient with the carrier, which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous phase or non-aqueous liquid; or as an oil-in-water liquid emulsion or a water-in-oil emulsion. The active ingredient may also be presented as a bolus, electuary or paste. Formulations for intra-tumoral or intra-lesional administration are particularly useful.

Details of pharmaceutically acceptable carriers, diluents and excipients and methods of preparing pharmaceutical compositions and formulations are provided in Remmingtons Pharmaceutical Sciences 18^{th} Edition, 1990, Mack Publishing Co., Easton, Pa., USA.

The present invention will now be further described with reference to the following examples, which are intended for the purpose of illustration only and are not intended to limit the generality of the subject invention as hereinbefore described.

The following methods and reagents are referred to in the Examples:

Cell cultures

The myeloid leukemic cell lines HL60, NB60, NB4, U937, K562 and KG1a were all grown in RPMI 1640 medium (Life Technologies, Paisley, United Kingdom) supplemented with 10% v/v fetal calf serum (Sear Laboratories International Crawley, United Kingdom), 100 U/mL penicillin, and 100 μg/mL streptomycin (Sigma Aldrich, Poole, United Kingdom) at 37° C. and 5% v/v CO₂. AML blasts were isolated from patient bone marrow aspirates and peripheral blood of eight patients at diagnosis or relapse. Patients were not receiving AML therapy at time of sampling. Blasts were isolated by Ficoll density centrifugation and were cultured in serum-free insulin transferrin selenium-positive (ITS+) medium (Life Technologies) containing 300 ng/mL stem cell factor (SCF) and 10 ng/mL interleukin 3 (IL3; R and D Systems, Abingdon, United Kingdom) and antibiotics. Blast purity ranged from 80% to 95%, as assessed by expression of CD34. Normal myeloblasts were isolated from cord blood or adult mobilized peripheral blood. Briefly, CD34⁺ cells were indirectly selected from the mononuclear cell preparations using antibody-coated magnetic beads (Miltenyi Biotec, United Kingdom).

Assays for Apoptosis and Differentiation in AML Cells

To determine the effects of ingenol-3-angelate (PEP005), cells were incubated for up to 5 days with medium alone or PEP005 at a range of concentrations from 0.2 nM to 20 μM. PEP005 was extracted from Euphorbia peplus and supplied as a 98.5% pure preparation by Peplin Limited (Brisbane, Australia) as a dry pellet and was made up to a stock of 20 μg/mL in acetone on a weekly basis. Stocks were stored at 4° C. and diluted into medium when required. Final acetone concentration was never more than 0.1% v/v. Apoptosis and differentiation were assessed after 1 to 5 days. Apoptosis was determined by several methods: cells were fixed and stained with propidium iodide and DNA content was revealed by flow cytometry, with apoptotic cells form a sub-G₀/G₁ peak (Telford et al, J Immunol Methods 172:1-16, 1994), the presence of activated caspase-3 was determined using a commercial kit based upon the cleavage of a fluorescent caspase-3 peptide substrate (Oncoimmunin, Columbus, Ohio); or the methyl-thiazol tetrazolium (MTT) assay was used as a marker of cell viability (Holleman et al, Blood 102:4541-4546, 2003).

Cell differentiation was determined by staining of cells with anti-CD11b antibody as a general early marker of myeloid differentiation. In studies with HL60 cells, PEP005-treated and 20 nM phorbol myristate (PMA)-treated (Sigma Aldrich) cells were also assessed for their ability to phagocytose complement-coated yeast, a marker of a fully differentiated myeloid cell, as previously described (Toksoz et al, Leukemia Res 6:491-498, 1982). The ingestion of three or more yeasts was taken as a positive result. In normal cord blood or adult bone marrow myeloblasts, loss of CD34 was taken as a marker of differentiation. Isotype-matched controls were used and all antibodies were from DAKO (Cambridge, United Kingdom). Differentiation was also induced by incubation of cells with 10 nM all-trans retinoic acid (ATRA; Sigma Aldrich). Cells were also used to prepare cytospins, which were then differentially stained using a commercial May-Grünwald-Ginesa stain (Diff-Kwik; Gamidor, Abingdon, United Kingdom) to identify cells with a blast or differentiated or apoptotic morphology. Slides were examined using a Zeiss III RS brightfield microscope equipped with a 40×/0.75 water-immersion objective (Carl Zeiss, Jena, Germany). Images were captured with a SPOT2 cameria (Diagnostic Instructions, Sterling Heights, Mich.) and analyzed using Image-Pro 4.0 software (Media Cybernetics, Silver Spring, Md.).

Protein Kinase C Activation and Expression Assays

PKC isoenzyme expression in leukemic cell lines was determined by Western blotting. Cells (0.5×10⁶) were lysed in lysis buffer (20 mM Tris [tris(hydroxymethyl-aminomethane]-HCl, pH 7.4, containing 150 mM NaCl, 0.5 mM EDTA [ethylenediaminetertraacetic acid], 1 mM dithiothreitol, 1 mM phenymethlsulfonylfuoride, 10 μg/mL of aprotinin, leupeptin, and pepstatin A, and 1% Triton-X-100). the lysate was spun at 1000 g for 10 minutes to isolate nuclei and was then combined 1:1 with sodium dodecyl sulfate (SDS) sample buffer and boiled for 5 minutes. The extracts were then analyzed by Western blotting using antibodies to the major isoenzymes found in myeloid cells, namely PKC-α, PKC-β, PKC-δ and PKC-ξ (all purchased from Santa Cruz Biotechnology, Santa Cruz, Calif.). Blots were developed using an enhanced chemiluminescence (ECL) method (Amersham Pharmacia, Buckinghamshire, United Kingdom).

PKC translocates from the soluble to the particulate fraction of cells when activated and this method was used to assess activation of PKC-δ by PEP005 and PMA. Briefly, cells were incubated for 10 minutes in medium alone or 20 nM PEP005 or 20 nM PMA, washed twice in phosphate buffered saline (PBS) and then lysed by homogenization in hypotonic lysis buffer in the absence of detergent. Lysis was checked using trypan blue uptake. Nuclei were isolated by centrifugation at 1000 g for 10 minutes. Centrifugation at 100,000 g for 45 minutes at 4° C. was used to isolate the cytosol (supernatant) and cell membrane (pellet) fractions. All were taken up in SDS sample buffer and analyzed for PKC-δ by Western blotting.

Caspase-3 phosphorylation

To determine if PEP005 induced caspase-3 phosphorylation, HL60 cells were incubated with 1.5×10⁷ Bq/mL ³²PO₄ (Amersham Pharmacia) in phosphate-free medium for 3 hours prior to addition of 20 nM PEP005 or PEP005 and 5 μM rottlerin (Calbiochem, Nottingham, United Kingdom). Cells were then lysed and caspase-3 was immunoprecipitated with an anti-caspase-3 antibody (BD Pharmingen, Oxford, United Kingdom) or an irrelevant isotype-matched control antibody (DAKO) and the immunoprecipitate was isolated using an anti-mouse immunoglobulin G (IgG) antibody (DAKO) and protein G-coated magnetic beads (microbeads; Miltenyi Biotech). The isolate was taken up in SDS sample buffer and run on a 10% w/v SDS-polyacrylamide gel electrophoresis (PAGE) gel and the labeled proteins were visualized using a phosphorimager.

PKC Transfection Studies

KG1a cells were transiently transfected with EGFP-tagged mouse PKC-δ subcloned into pEGFP-N1 plasmid (available from National Cancer Institute [NCI], Bethesda, Md.) using an Amaxa nucleofection apparatus (Amaxa, Koeln, Germany). Transfection efficiency was approximately 35% as judged by fluorescence-activated cell sorter (FACS) analysis and cells were treated with PEP005 (0.2 μM-20 μM) 24 hours after transfection. Cell viability in EGFP-positive cells was assessed by analysis of cell morphology (forward scatter and side scatter profile) by FACS and loss of viability confirmed in the total cell culture by MTT assay after 3 days. Briefly, 24 hours after transfection, 2×10⁴ cells were plated in five wells in 96-well plates and exposed to 0, 0.2, 2 and 2.0 μM PEP005. At 72 hours, 20 μL MTT substrate at 5 mg/mL was added and plates were incubated at 37° C. After 3 hours, 150 μL media was removed and replated with 200 μL dimethyl sulfoxide (DMSO). Absorbance at an optical density (OD) of 550 nm was read on a Biotech plate reader (Amersham Pharmacia) and corrected for absorbance obtained from blank media controls.

Immunofluorescen Imaging of PKC-δ-GFP Activation

KG1a cells were transfected with PKC-δ-EGFP or pEGFP-N1 (vector control). At 24 hours after transfection, cells were treated with 0, 0.2, 2 and 20 μM PEP005 and cytospins prepared after 15 minutes. Slides were fixed with 2% v/v paraformaldehyde in PBS for 20 minutes, rinsed briefly in PBS and mounted in Vectashield containing DAPI (4.6 diamidino-2-phenylindole; Vector Laboratories, Burlingame, Calif.). Slides were examined using a Leica Fluorescence microscope (Leica, Heidelberg, Germany) fitted with ×60 oil immersion objective. Images were captured using a Hamanatsu C4742-95 camera (Grafter Imaging, Austin, Tex.) and analyzed using OpenLab 3.1 software (Improvision, Coventry, United Kingdom).

Statistics

Data presented here represent a minimum of three experiments and where appropriate, data are expressed as means plus or minus SD. Statistical significance was assessed by Student t test and a P value less than 0.05 was taken as a significantly different value.

### EXAMPLE 1

### PEP005 has Anti-Leukemic Effects Against Cell Lines and Primary AML Blasts

PEP005 is a small molecule activator of the eight classical PKC isoenzymes (Kedei et al, 2004 supra). PEP005 has already been shown to have anti-neoplastic potential against skin cancers, and an initial screen of the cytotoxic effects of PEP005 against other cancer cell types revealed potent effects on leukemic cell lines. The anti-leukemic potential of PEP005 was therefore investigated further.

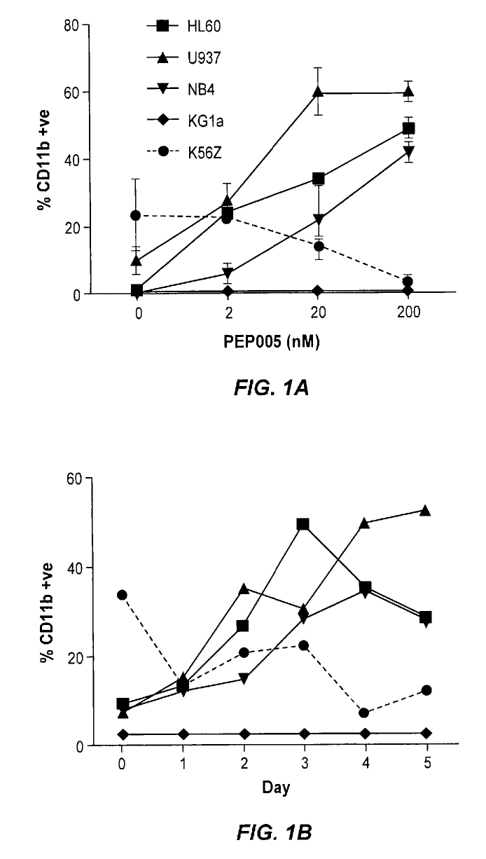

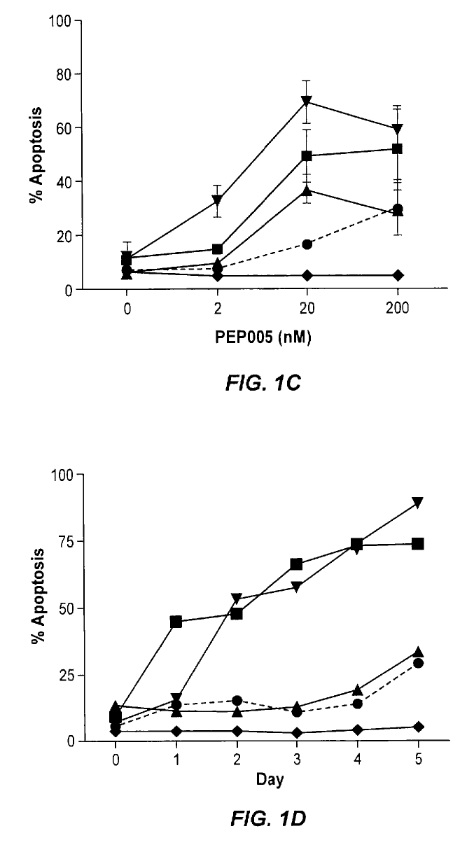

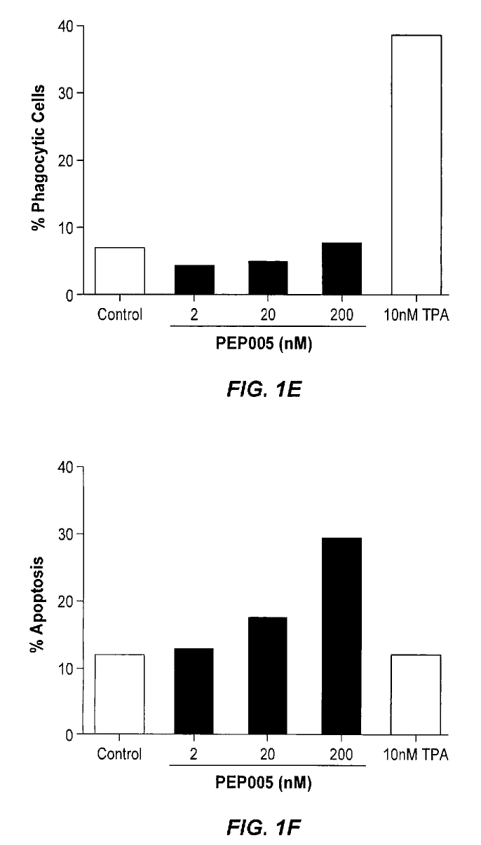

There were 5 myeloid leukemia cell lines treated with PEP005 and differentiation and apoptosis were determined. Differentiation was initially assessed by gain of CD11b expression (FIG. 1A). Apoptosis was measured by FACS analysis of sub-G I DNA (FIG. 1C) and caspase-3 activation (data not shown). There were 3 cell lines (HL60, NB4, and U937) induced to express CD11b (FIG. 1A) and enter apoptosis (FIG. 1C) in response to nanomolar PEP005 concentrations, with optimal effects at 20 nM. K562 cells also responded to PEP005 but were less sensitive, with the maximal response seen with 2 μM PEP005; they only entered apoptosis and did not increase expression of CD11b (FIG. 1A-B). KG1 a cells were resistant to PEP005 and did not enter apoptosis or show CD11b expression even at 20 μM PEP005.

Kinetic studies (FIGS. 1B,D) suggested that some cell cultures, such as NB4 and HL60 showed a mixed response, with up to half of the cells entering apoptosis within the first 1 to 2 days of treatment and the remainder expressing CD11b. Other lines such as U937 expressed CD11b as the predominant response before entering apoptosis. The differentiation data gained using CD11b as a marker were, therefore, difficult to interpret. However, gain of CD11b is an early step in the differentiation process and to determine if PEP005 differentiated promyeloid cells into functional end cells (monocytes or neutrophils), HL60 cells were treated with PEP005 or PMA and measured their ability to phagocytose yeast after 3 days of treatment. While PMA induced differentiation of HL60 cells toward phagocytes (FIG. 1E) with no increase in apoptosis (FIG. 1F), PEP005 treatment did not lead to production of phagocytic cells and apoptosis was the dominant effect. It was concluded that induction of apoptosis was the predominant effect of PEP005.

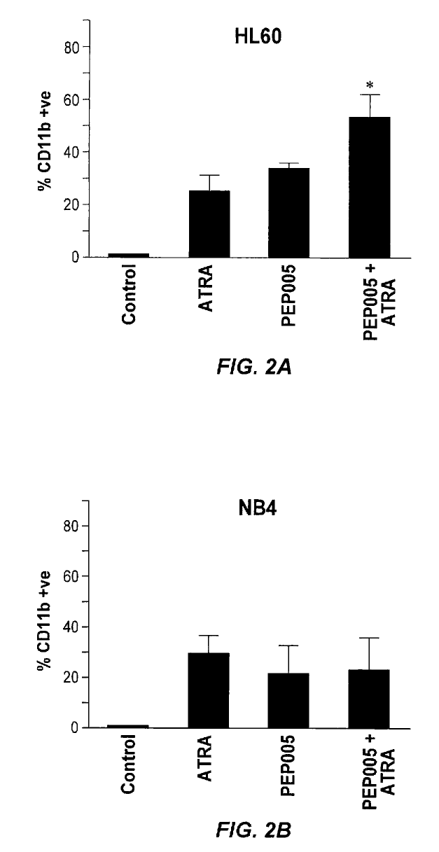

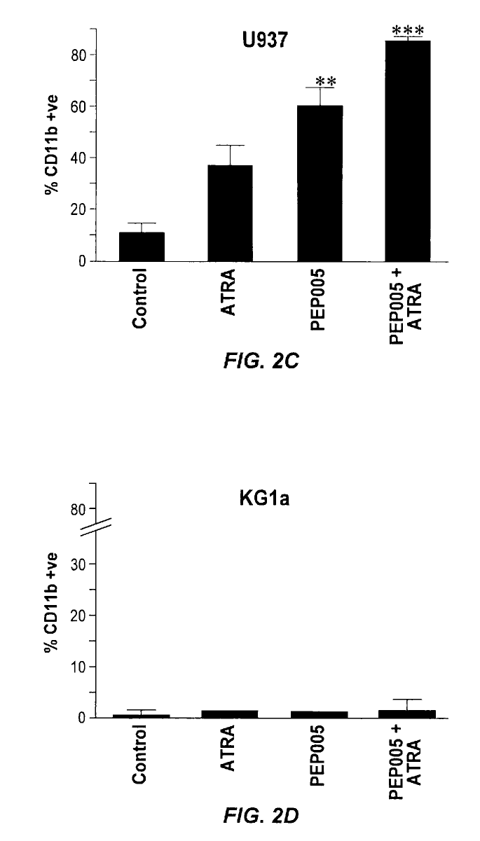

It was noted that the PEP005-responsive cell lines were those lines that could also be induced to differentiate by all-trans retinoic acid (ATRA), an agent used in differentiation therapy of acute promyelocytic leukemia (APL). To determine if the responses to PEP005 and ATRA were interrelated, cells were treated with ATRA and PEP005 alone and in combination and measured the differentiation response. PEP005 was able to synergize with ATRA in two of the cell lines (HL60 and U937) but not in NB4 (FIG. 2) or K562 cells. KG1a did not respond to ATRA or PEP005.

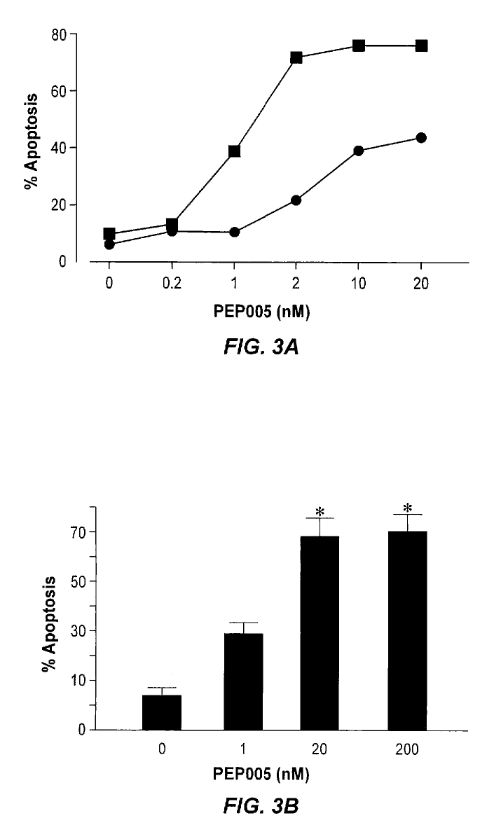

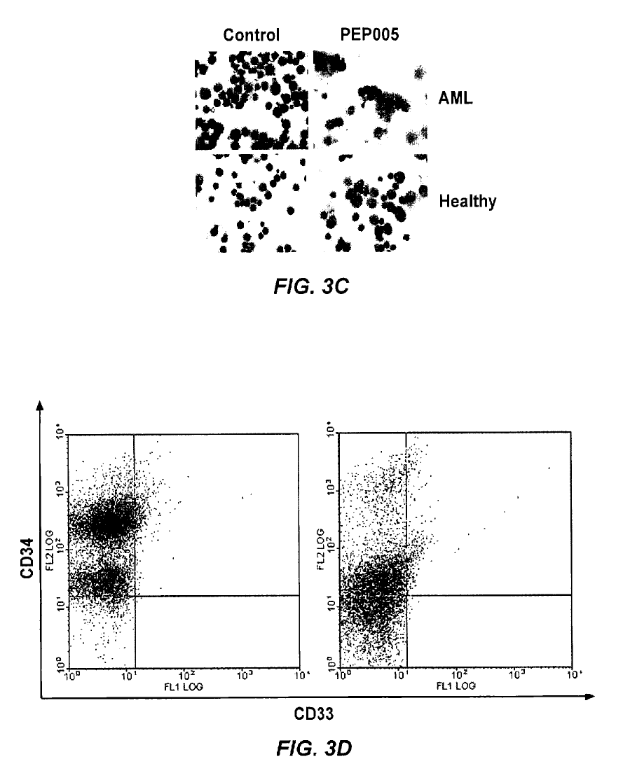

Blasts isolated from the bone marrow of eight patients diagnosed with AML were also treated with PEP005, and seven of these were induced to enter apoptosis. Apoptosis was measured by FACS analysis of sub-G1 DNA and caspase-3 activation (FIG. 3A). After 48 hours of treatment with 20 nM PEP005, few viable AML blast cells remained, and the shrunken size and condensed nuclear morphology characteristic of apoptosis were predominant (FIG. 3C, top panels). All seven responsive AML samples showed similar sensitivity to PEP005, with apoptosis induced at concentrations as low as 2 nM and with maximal effect seen at 10 to 20 nM PEP005 (FIG. 3A-B). The major difference in response seen between the AML samples was in the level of apoptosis induced, and at 20 nM PEP005, the values for induction of apoptosis ranged from 56% to 95%. Normal CD34⁺ myeloblasts isolated from cord blood (FIG. 3C) and from adult marrow acquired after stem cell mobilization therapy were also exposed to PEP005. Induction of apoptosis was not seen at concentrations of PEP005 that were effective against cell lines and AML blast cells, and cytotoxicity did not occur even at 200 nM PEP005 (FIG. 3C, top panels). Interestingly, the normal myeloblasts were induced to differentiate, as indicated by loss of CD34 and progression to promyelocyte- and myelocyte-like morphologies, in response to PEP005, and this effect was seen at doses of 20 nM to 2.0 μM PEP005 (FIG. 3C, lower panel).

These observations indicate a potentially broad therapeutic window for PEP005 that spans at least two-log concentrations.

### EXAMPLE 2

### PEP005 Responsiveness Correlates with PKC-δ Expression

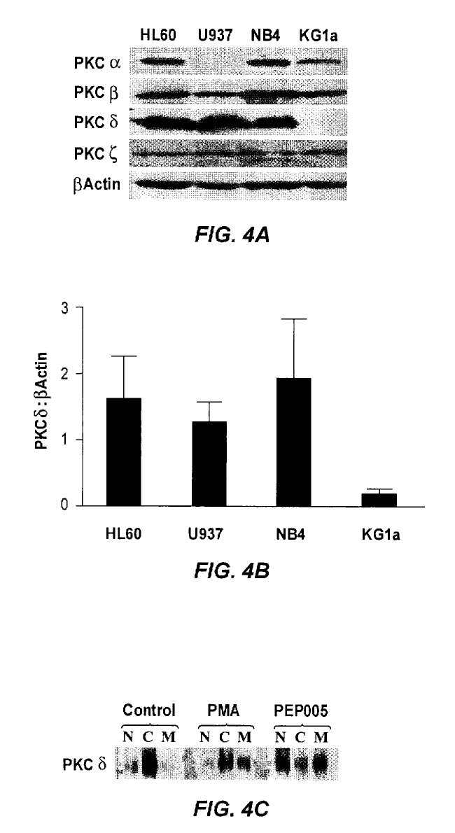

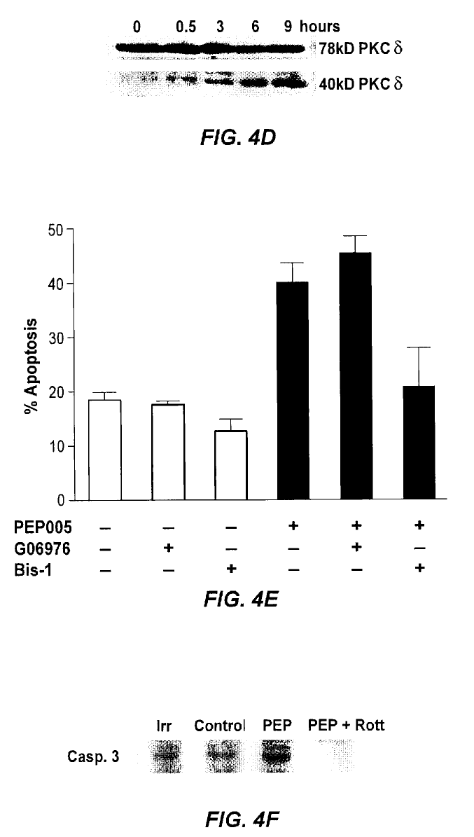

It was determined whether the varied responsiveness seen in Example 1 of leukemic cell lines to PEP005 was related to differential expression of PKC isoenzymes. Expression of the four isoenzymes that predominate in myeloid cells, namely PKC-α, -β, -δ and -ξ was measured. There was no consistent association between PEP005 responsiveness and expression of PKC-α, PKC-β or PKC-ξ (FIG. 4A). However, expression of PKC-δ did correlate with PEP005 sensitivity with good levels of this isoenzyme expressed in HL60, U937 and NB4 and no significant expression detected in KG1a (FIG. 4B). In addition, PEP005 induced activation of PKC-δ in HL60 cells, detected by translocation of the enzyme from the cytosol to the membrane fraction of cells (FIG. 4C). Interestingly, initial translocation of PKC-δ was to the nuclear and cell membranes following treatment with PEP005, whereas PMA induced translocation only to the cell membrane (FIG. 4C). This differential effect of PEP005 and PMA on PKC-δ subcellular location was also reported in COS cells transfected with PKC-δ (Kedei et al, C. Cancer Res. 64:4243-3255, 2004). PKC-δ is known to undergo proteolysis of caspase-3 to release a pro-apoptotic 40-kDa catalytic fragment and it was, therefore, determined if PEP005 treatment resulted in the production of the 40-kDa fragment of PKC-δ. FIG. 4D shows that the 40-kDa fragment was produced rapidly in HL60 cells. The likelihood that the pro-apoptotic activity of PEP005 was mediated through activation of PKC-δ was further supported by the observation that PEP005-induced apoptosis of HL60 cells was inhibited by the broad range. PKC inhibitor bisindolylmaleimide I (Bis-I, FIG. 4E), but not by Go6976, which is an inhibitor of the classical PKC isoenzymes PKC-α and -β (Nowak, J Biol Chem. 277:43377-43388, 2002). Interestingly, the differentiating actions of ATRA on leukemic cells have been shown to be in part mediated via activation of PKC-δ and phosphorylation of retinoid acid receptor α (RARα) (Kambhampati et al, J Biol Chem 278:32544-32551, 2003). This may explain why PEP005-induced partial differentiation was seen only in the ATRA-responsive cell lines. To investigate the possible pro-apoptotic mode of action of PKC-δ, the effect of PEP005 treatment on caspase-3 phosphorylation was determined. It has been reported that PKC-δ was able to phosphorylate and activate caspase-3 in monocytic cells (Voss et al, J Biol Chem 280:17371-17379, 2005). FIG. 4F shows that PEP005 was able to induce caspase-3 phosphorylation and that this was ablated by the PKC-δ inhibitor rottlerin.

### EXAMPLE 3

### Expression of PKC-δ in KG1a Cells Confers Responsiveness to PEP005

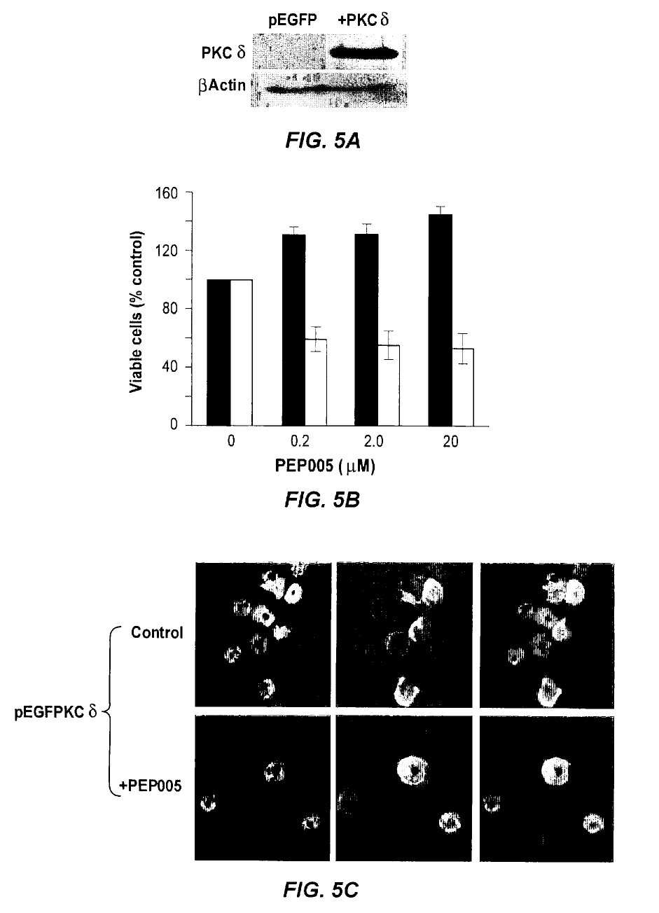

To confirm the key role played by PKC-δ in mediating the effects of PEP005. KG1a cells were transfected with EGFP-tagged PKC-δ and 24 hours later were treated with PEP005 for 3 days. Transfection was confirmed by FACS analysis and transfection efficiency was approximately 35%. Expression of PKC-δ at the protein level was confirmed by Western blotting 24 hours after transfection (FIG. 5A). Importantly, exposure of EGFP-positive PKC-δ-expressing KG1a cells to PEP005 resulted in apoptosis, assessed by increased forward scatter and decreased side scatter by FACS, at doses commensurate with those inducing apoptosis in HL60, U937, and NB4 cells. In contrast, EGFP-positive cells transfected with control vector were resistant to PEP005-induced apoptosis even at 20 μM PEP005 (FIG. 5B). Loss of viability was confirmed in the total cell population by MTT assay.

Finally, fluorescence microscopy confirmed that PEP005-induced apoptosis of EGFP-PKC-δ-expressing KG1a cells was preceded by the activation of PKC-δ. FIG. 5C shows that prior to exposure to PEP005, EGFP-PKC-δ was located predominantly in the cytosol of KG1a cells but that PEP005 treatment induced its translocation to the nucleus and perinuclear region. Furthermore, it was shown previously that translocation of PKC-δ to the nucleus is associated with induction of apoptosis in human neutrophils and T-cells (Scheel-Toellner et al, Eur J Immunol 29:2603-2612, 1999) and that key substrates of this isoenzyme involved in the induction of apoptosis include nuclear lamin B (Cross et al, Oncogene 19:2331-2337, 2000).

It is concluded, therefore, that PEP005 mediates selective and potent anti-neoplastic actions against AML cells that are mediated via the activation of PKC-δ and its translocation to the nucleus.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

The sequence listing attached is hereby incorporated by reference in this application. (See Apr. 13, 2007 sequence listing as filed.txt created on Apr. 13, 2007 that is 9,855 bytes).

1. A method comprising;
a) administering to a subject with cancer an anti-cancer effective amount of an ingenol angelate or pharmaceutically acceptable salt thereof, wherein said cancer contains cells that produce high levels or activities of a PKC or other component in the PKC signalling pathway in order to treat said subject with cancer.

2. The method of claim 1 wherein the level or activity of PKC or other component in the PKC signaling pathway is determined prior to treatment with said ingenol angelate.

3. The method of claim 1 wherein said PKC is PKC-δ.

4. The method of claim 1 wherein said ingenol angelate is PEP005 or a derivative, homolog, isomer or pharmaceutically acceptable salt thereof.

5. The method of claim 1 wherein said subject is a human.

6. The method of claim 1 wherein said cancer is a hematological-based cancer.

7. The method of claim 6 wherein said haematological-based cancer is a myeloid leukemia.

8. The method of claim 7 wherein said myeloid leukemia is AML or CML.

9. A method comprising;
a) determining the levels or activity of a PKC or other component in the PKC signalling pathway in one or more cancer cells of a subject; and
b) wherein said cancer cells have low to zero levels of PKC or other component in the PKC signaling pathway, contacting said cancer cells with the PKC or other component in the PKC signaling pathway or a genetic means for generating same and an ingenol angelate or a pharmaceutically acceptable salt thereof in order to treat said subject with cancer.

10. The method of claim 9 wherein said PKC is PKC-δ.

11. The method of claim 9 wherein said ingenol angelate is PEP005 or a derivative, homolog, isomer or pharmaceutically acceptable salt thereof.

12. The method of claim 9 wherein said subject is a human.

13. The method of claim 9 wherein said cancer is a hematological-based cancer.

14. The method of claim 13 wherein said hematological-based cancer is a myeloid leukemia.

15. The method of claim 14 wherein said myeloid leukemia is AML or CML.

16. A method comprising;
contacting cancer cells with PKC or other component in the PKC signalling pathway and an ingenol angelate or a derivative, homolog, isomer or pharmaceutically acceptable salt thereof for a time and under conditions sufficient for the ingenol angelate to be toxic to said cells in a subject with a haematological-based cancer.

17. The method of claim 16 wherein said PKC is PKC-δ.

18. The method of claim 16 wherein said ingenol angelate is PEP005 or a derivative, homolog, isomer or pharmaceutically acceptable salt thereof.

19. The method of claim 16 wherein said hematological-based cancer is a myeloid leukemia.

20. The method of claim 19 wherein said myeloid leukemia is AML or CML.

21. A method comprising;
determining the levels or activity of a PKC or other component in the PKC signalling pathway in one or more cancer cells of a subject, wherein the absence of low levels of PKC or other component in the PKC signalling pathway compared to a control indicates that the cancer cells are less sensitive to an ingenol angelate.

22. The method of claim 21 wherein said PKC is PKC-δ.

23. The method of claim 21 wherein said ingenol angelate is PEP005 or a derivative, homolog, isomer or pharmaceutically acceptable salt thereof.

24. The method of claim 21 wherein said subject is a human.

25. The method of claim 21 wherein said cancer is a hematological-based cancer.

26. The method of claim 25 wherein said hematological-based cancer is a myeloid leukemia.

27. The method of claim 26 wherein said myeloid leukemia is AML or CML.

28. A composition comprising;
an ingenol angelate and a PKC or genetic means for producing same for the treatment of a subject having cancer.

29. A composition comprising;
(a) a first part comprising an ingenol angelate or a derivative, homolog, isomer or pharmaceutically acceptable salt thereof; and
(b) a second part comprising a PKC or other component of the PKC signalling pathway or a genetic means of generating same.

30. The composition of claim 29 wherein said ingenol angelate is PEP005.

31. The composition of claim 29 wherein said PKC is PKC-δ.

32. A method comprising:
a) isolating cancer cells from a subject; and
b) subjecting said cells to PKC-δ-or other component-detection means in order to screen for the presence, level or activity of PKC or other component of the PKC signalling pathway.

33. The method of claim 32 wherein said PKC is PKC-δ.

34. The method of claim 32 wherein said cells are hematological-based cancer cells.

35. The method of claim 34 wherein said hematological-based cancer cells are myeloid leukemia cells.

36. The method of claim 35 wherein said myeloid leukemia cells are AML or CML.
